(19) United States
(12) Patent Application Publication
(10) Pub. No.: US 2024/0321407 A1
(43) Pub. Date: Sep. 26, 2024

(54) MACHINE LEARNING PLATFORM FOR PREDICTED EFFICACY OF UNTESTED PHARMACEUTICALS

(71) Applicant: MCKINSEY & COMPANY, INC., New York, NY (US)

(72) Inventors: Christoforos Anagnostopoulos, Athens (GR); Maren Eckhoff, London (GB)

(21) Appl. No.: 18/586,129

(22) Filed: Feb. 23, 2024

Related U.S. Application Data
(60) Provisional application No. 63/486,917, filed on Feb. 24, 2023.

(51) Int. Cl. G16C 20/30; G16C 20/70

(52) U.S. Cl. CPC G16C 20/30; G16C 20/70

(57) ABSTRACT

Methods and systems for predicting an efficacy value of an untested pharmaceutical for treating a malady. Machine learning models may be trained using sets of pharmaceutical-pathway weight impact scores and patient data to predict the efficacy of an untested pharmaceutical in treating a particular malady. The machine learning models may also be tailored to specific patients based upon characteristics in common with patients in the set of patient data.

### RELATED APPLICATION

This application claims the benefit of U.S. Provisional Application No. 63/486,917 (filed on Feb. 24, 2023), which is incorporated in its entirety by reference herein.

### TECHNICAL FIELD

The present disclosure generally relates to machine learning algorithms, techniques, platforms, methods, and systems for predicting the efficacy of a pharmaceutical prior to clinical testing.

### BACKGROUND

Traditionally, the efficacy of a pharmaceutical in treating a particular malady prior to clinical testing has been predicted using pre-clinical data such as animal models, or computational simulations of the action of the pharmaceutical. However, such predictions are often invalidated upon subsequent clinical testing. Clinical evidence of efficacy of other pharmaceuticals that had already been approved or clinically tested has not been used effectively to estimate the efficacy the untested pharmaceutical in treating the malady, even when said other pharmaceuticals were similar in molecular structure or molecular action to the untested pharmaceutical.

Accordingly, herein, in order to address the aforementioned issues, systems and methods for predicting an efficacy value of an untested pharmaceutical for treating a malady are disclosed.

### SUMMARY

In some embodiments, a computer-implemented method for predicting an efficacy value of an untested pharmaceutical for treating a malady may be provided. The method may be implemented via one or more local or remote processors, servers, memory units, mobile devices, wearables, and/or other electronic or electrical components. In one instance, the method may include: (1) receiving, by one or more processors, a set of training data which may include: (i) a set of pharmaceutical-pathway weight impact scores, the set of pharmaceutical-pathway weight impact scores may include a weighted impact score of each previously human tested pharmaceutical in a set of previously human tested pharmaceuticals for treating the malady across each human biological molecule-protein pathway in a set of human biological molecule-protein pathways and/or (ii) a set of patient data, the set of patient data may include (a) a set of characteristics of each patient, (b) a set of previously human tested pharmaceuticals taken by each patient in treatment of the malady, and/or (c) one or more observed clinical outcomes that can be used to estimate the efficacy of the previously human tested pharmaceutical received by each patient; (2) training, by one or more processors, a machine learning model using the set of training data, the machine learning model configured to output a predicted efficacy value of an untested pharmaceutical in treating the malady; (3) receiving, by the one or more processors, a weighted impact score of an untested pharmaceutical across each human biological molecule-protein pathway in the set of human biological molecule-protein pathways; (4) analyzing, by the one or more processors using the trained machine learning model, a first set of input data to generate a predicted efficacy value of the untested pharmaceutical in treating the malady, the first set of input data may include: (i) the weighted impact score of the untested pharmaceutical across each human biological molecule-protein pathway in the set of human biological molecule-protein pathways and/or (ii) the set of patient data; and/or (5) communicating, by the one or more processors to a client device, the predicted efficacy value of the untested pharmaceutical in treating the malady. The method may include additional, less, or alternate actions, including those discussed elsewhere herein.

In other embodiments, a computer system for predicting an efficacy value of an untested pharmaceutical for treating a malady may be provided. The computer system may include, or be configured to work with, one or more local or remote processors, servers, memory units, mobile devices, wearables, and/or other electronic or electrical components. In one instance, the computing system may include one or more processors and/or associated transceivers, and/or a non-transitory program memory coupled to the one or more processors and/or storing executable instructions that, when executed by the one or more processors, cause the computer system to: (1) receive a set of training data which may include: (i) a set of pharmaceutical-pathway weight impact scores, the set of pharmaceutical-pathway weight impact scores may include a weighted impact score of each previously human tested pharmaceutical in a set of previously human tested pharmaceuticals for treating the malady across each human biological molecule-protein pathway in a set of human biological molecule-protein pathways and/or (ii) a set of patient data, the set of patient data may include (a) a set of characteristics of each patient, (b) a set of previously human tested pharmaceuticals taken by each patient in treatment of the malady, and/or (c) one or more observed clinical outcomes that can be used to estimate the efficacy of the previously human tested pharmaceutical received by each patient; (2) train a machine learning model using the set of training data, the machine learning model configured to output a predicted efficacy value of an untested pharmaceutical in treating the malady; (3) receive a weighted impact score of an untested pharmaceutical across each human biological molecule-protein pathway in the set of human biological molecule-protein pathways; (4) analyze, using the trained machine learning model, a first set of input data to generate a predicted efficacy value of the untested pharmaceutical in treating the malady, the first set of input data may include: (i) the weighted impact score of the untested pharmaceutical across each human biological molecule-protein pathway in the set of human biological molecule-protein pathways and/or (ii) the set of patient data; and/or (5) communicate, to a client device, the predicted efficacy value of the untested pharmaceutical in treating the malady. The computer system may be configured to include additional, less, or alternate functionality, including that discussed elsewhere herein.

In yet other embodiments, a non-transitory computer-readable medium for predicting an efficacy value of an untested pharmaceutical for treating a malady may be provided. The executable instructions, when executed by one or more processors of a computer system, may cause the computer system to: (1) receive a set of training data which may include: (i) a set of pharmaceutical-pathway weight impact scores, the set of pharmaceutical-pathway weight impact scores may include a weighted impact score of each previously human tested pharmaceutical in a set of previously human tested pharmaceuticals for treating the malady across each human biological molecule-protein pathway in a set of human biological molecule-protein pathways and/or (ii) a set of patient data, the set of patient data may include (a) a set of characteristics of each patient, (b) a set of previously human tested pharmaceuticals taken by each patient in treatment of the malady, and/or (c) one or more observed clinical outcomes that can be used to estimate the efficacy of the previously human tested pharmaceutical received by each patient; (2) train a machine learning model using the set of training data, the machine learning model configured to output a predicted efficacy value of an untested pharmaceutical in treating the malady; (3) receive a weighted impact score of an untested pharmaceutical across each human biological molecule-protein pathway in the set of human biological molecule-protein pathways; (4) analyze, using the trained machine learning model, a first set of input data to generate a predicted efficacy value of the untested pharmaceutical in treating the malady, the first set of input data may include: (i) the weighted impact score of the untested pharmaceutical across each human biological molecule-protein pathway in the set of human biological molecule-protein pathways and/or (ii) the set of patient data; and/or (5) communicate, to a client device, the predicted efficacy value of the untested pharmaceutical in treating the malady. The instructions may direct additional, less, or alternate functionality, including that discussed elsewhere herein.

The present disclosure may include improvements in computer functionality or in improvements to other technologies at least because the disclosure herein discloses systems and methods predicting the efficacy of an untested pharmaceutical for treating a malady. The systems and methods herein may train machine learning models using input data vectors (e.g., sets of pharmaceutical-pathway weight impact scores, sets of patient data, untreated patient data, etc.) to generate an efficacy value of the untested pharmaceutical in treating the malady based upon the relationship between previously human tested pharmaceuticals, human biological molecule-protein pathways, and patient characteristics. For example, when deployed on the underlying system, the machine learning models allow the systems and methods of the present disclosure to execute with fewer iterations, and use fewer computing resources, than prior art related systems and methods, at least because such prior art systems would require manual data entry, data storage, and/or implementation, all of which result in greater memory usage and processor utilization.

Additional improvements may also include determined corollary and/or causal outputs. For example, the system, utilizing the machine learning models, may be able to determine, predict, and/or propose explanations as to why a predicted efficacy of an untested pharmaceutical is high or low based upon the determined relationships of the untested pharmaceutical to the various human biological molecule-protein pathways involved.

Similarly, the present disclosure describes improvements in the functioning of the computer itself or “any other technology or technical field” because the data generated (e.g., the predicted efficacy values of the untested pharmaceutical) described herein allows the underlying computer system to utilize less processing and memory resources compared to prior art systems and methods. This is at least because the machine learning models can generate and/or determine data of a predictive function of a pharmaceutical's efficacy in treating a malady without the need for various tests and/or empirical computer simulation across a wide range of tests using multiple compute cycles and data. Therefore, use of the machine learning models results in fewer compute cycles, or otherwise iterations, that has less of an impact on the underlying computing device compared to previous prior art systems and methods. In addition, the systems and methods of the present disclosure improve over the prior art at least because prior art systems and methods require an empirical or trial-and-error approach that can involve real-world trials that can result in, and require, large database and memory utilization and processor usage to arrive at a similar real-world or simulated results that has a same or similar result.

In addition, the present disclosure relates to improvement to other technologies or technical fields at least because the systems and methods of the present disclosure provide a robust, efficient, and comparable model that can be used to improve the efficiency and performance of several downstream pharmaceutical discovery, development, and/or manufacturing tasks. This may be performed, for example, by a machine learning model that is determined or otherwise generated with based upon potential pharmaceutical candidates for treating the malady and/or families of pharmaceutical compounds that might be developed in treating the malady. The machine learning model may be deployed on an underlying computing device or system, thereby, improving its accuracy and prediction in performing pharmaceutical discovery, development, and/or manufacturing tasks as described herein.

Still further, the present disclosure includes specific features other than what is well-understood, routine, conventional activity in the field, and/or otherwise adds unconventional steps that confine the disclosure to a particular useful application (e.g., systems and methods for predicting an untested pharmaceutical's efficacy in treating a malady based upon the machine learning model) which can be used, for example, for the effective and efficient output of an efficacy value of an untested pharmaceutical, which may be used or applied for pharmaceutical discovery, development, and/or manufacturing applications. Example applications may include: (i) prioritizing which compounds to further research in a clinical development strategy plan, (ii) determining how to best combine multiple candidate pharmaceuticals based upon a patient's likelihood response to each of those candidate pharmaceuticals, (iii) determining which previously human tested pharmaceuticals should receive further clinical testing in order to determine the comparative predicted efficacy of the untested pharmaceutical, and/or the like.

Advantages will become more apparent to those of ordinary skill in the art from the following description of the preferred embodiments, which have been shown and described by way of illustration. As will be realized, the present embodiments may be capable of other and different embodiments, and their details are capable of modification in various respects. Accordingly, the drawings and description are to be regarded as illustrative in nature and not as restrictive.

### BRIEF DESCRIPTION OF THE DRAWINGS

The FIGs. described below depict various embodiments of the systems and methods disclosed herein. It should be understood that the FIGs. depict illustrative embodiments of the disclosed systems and methods, and that the FIGs. are intended to be exemplary in nature. Further, wherever possible, the following description refers to the reference numerals included in the following FIGs., in which features depicted in multiple FIGs. are designated with consistent reference numerals.

There are shown in the drawings arrangements which are presently discussed, it being understood, however, that the present embodiments are not limited to the precise arrangements and instrumentalities shown, wherein:

FIG. 1 depicts exemplary components, apparatuses, and devices used by devices and systems for implementing an untested pharmaceutical efficacy value prediction system;

FIG. 2 depicts an exemplary computing environment including components, apparatuses, and devices for implementing the untested pharmaceutical efficacy value prediction system;

FIG. 3 depicts an exemplary input vector for the untested pharmaceutical efficacy value prediction system with example input data;

FIG. 4A depicts exemplary input data that may be used to generate the exemplary input vector of the untested pharmaceutical efficacy value prediction system;

FIG. 4B further depicts exemplary input data that may be used to generate the exemplary input vector of the untested pharmaceutical efficacy value prediction system;

FIG. 4C further depicts exemplary input data that may be used to generate the exemplary input vector of the untested pharmaceutical efficacy value prediction system;

FIG. 4D further depicts exemplary input data that may be used to generate the exemplary input vector of the untested pharmaceutical efficacy value prediction system;

FIG. 4E further depicts exemplary input data that may be used to generate the exemplary input vector of the untested pharmaceutical efficacy value prediction system;

FIG. 5 depicts exemplary machine learning modules;

FIG. 6 depicts an exemplary flowchart representative of example methods, logic, and instructions for implementing the untested pharmaceutical efficacy value prediction system;

FIG. 7 depicts an exemplary flowchart representative of example methods, logic, and instructions for training and testing the machine learning models;

FIG. 8 depicts an exemplary computer-implemented method for predicting an efficacy value of an untested pharmaceutical for treating a malady; and

The figures depict the present embodiments for purposes of illustration only. One skilled in the art will readily recognize from the following discussion that alternate embodiments of the structures and methods illustrated herein may be employed without departing from the principles of the invention described herein.

### DETAILED DESCRIPTION

Embodiments of the present description relate to computing systems and methods for predicting an efficacy value of an untested pharmaceutical for treating a malady. In some embodiments, machine learning models may be trained using sets of pharmaceutical-pathway weight impact scores and patient data to predict the efficacy of an untested pharmaceutical in treating a particular malady.

Sets of pharmaceutical-pathway weight impact scores may include correlations between pharmaceuticals that have been previously tested in treating the malady in addition to human biological molecule-protein pathways. These correlations may describe the impact by which those pharmaceuticals impact those specific pathways. Additionally, because these pharmaceuticals were previously tested in treating the malady, those pharmaceuticals have been associated with observed clinical outcomes on patients and/or have an estimated efficacy value based upon observed clinical outcomes in treating the malady (e.g., via randomized clinical trials, causal inference on observational data, and/or the like). Therefore, relationships may be made between pharmaceuticals with positive observed clinical outcomes (e.g., many years of survival, several months without symptoms, etc.) in treating the malady and impact by which the human biological molecular-protein pathways are affected by those pharmaceuticals. Thus, a prediction can be made on an untested pharmaceutical's unknown efficacy value in treating the malady if the untested pharmaceutical's impact on the various human biological molecule-protein pathways is known. In some embodiments, the set of pharmaceutical-pathway weight impact scores may be extracted as data summaries obtained from one or more knowledge graphs which illustrate known interactions and/or associations between proteins and/or molecules that are related to biological entities such as genes and patient phenotypes (in some examples, these may also be referred to as “protein-protein interaction knowledge graphs”). Additionally, or alternatively, a weighted impact score may comprise an impact score that includes an integer or Boolean value (0 or 1). In such aspects, an impact score need not be a weighted impact score, and such an impact score may be used in addition to, or in alternative to, one or more weighted impact scores.

However, using only the sets of pharmaceutical-pathway weight impact scores does not account for the plethora of other unknown variables between two individuals suffering from a particular malady. Even two individuals with the same malady with very similar demographics may respond differently to treatment based upon various factors (e.g., other prescribed medications, dietary habits, genetic predispositions, etc.). As such, the data sets and relationships need to also include and account for sets of patient data (e.g., data related to patients ailed by the malady such as medical history, family medical history, demographics, other medications currently prescribed, living conditions, other comorbidities, markers of general health such as body-mass-index, age etc.). Incorporating patient data into the machine learning model should make the resulting predicted efficacy values more accurate to a real efficacy value that may be verified via clinical testing.

Further, the machine learning model may be tailored to a specific patient suffering from the malady. To accomplish this, the set of patient data must be reduced to a subset of patients with characteristics that best match the specific patient.

It should be noted that the malady may be characterized by some end point in the data, often assessed using one or more clinical outcomes (e.g., length of survival). Furthermore, it should also be appreciated that the efficacy prediction models need not only apply to one malady at a time, and that efficacy values of the untested pharmaceutical may be generated across a set of maladies provided they are all assessed using the same end point in the data and/or clinical outcome(s) (e.g., length of survival).

### Exemplary Machine Learning Techniques

The present embodiments may involve, inter alia, the use of cognitive computing, predictive modeling, machine learning, causal inference and/or other modeling techniques and/or algorithms. In particular, a set of pharmaceutical-pathway weight impact scores, a set of previously human tested pharmaceutical data, human biological molecule-protein pathways, a set of patient data, untreated patient data, and/or the like may be input into one or more machine learning programs described herein that are trained and/or tested to predict an efficacy value of an untested pharmaceutical for treating a malady.

In certain embodiments, the systems, methods, and/or techniques discussed herein may use heuristic engines, algorithms, machine learning, cognitive learning, deep learning, combined learning, predictive modeling, and/or pattern recognition techniques. For instance, a processor and/or a processing element may be trained using supervised or unsupervised machine learning, and the machine learning program may employ a neural network, which may be a convolutional neural network, a deep learning neural network, and/or a combined learning module or program that learns in two or more fields or areas of interest. Machine learning may involve identifying and/or recognizing patterns in existing data in order to facilitate making predictions, estimates, and/or recommendations for subsequent data. Models may be created based upon example inputs in order to make valid and reliable predictions for novel inputs.

Additionally or alternatively, the machine learning programs may be trained and/or tested by inputting sample data sets or certain data into the programs, such as a set of pharmaceutical-pathway weight impact scores, a set of previously human tested pharmaceutical data, human biological molecule-protein pathways, a set of patient data, and/or known resulting data (e.g., one or more predicted observed clinical outcomes of previously human tested pharmaceutical data and/or one or more actually observed clinical outcomes of prior untested pharmaceutical data). The machine learning programs may utilize deep learning algorithms that may be primarily focused on pattern recognition and may be trained after processing multiple examples. The machine learning programs may include Bayesian program learning (BPL), voice recognition and synthesis, image or object recognition, optical character recognition, and/or natural language processing-either individually or in combination. The machine learning programs may also include natural language processing, semantic analysis, automatic reasoning, and/or machine learning.

In supervised machine learning, a processing element may be provided with example inputs (e.g., the set of patient data) and their associated outputs (e.g., the observed clinical outcomes of patients from the set of patient data), and may seek to discover a general rule that maps inputs to outputs, so that when subsequent novel inputs are provided the processing element may, based upon the discovered rule, accurately predict the correct output. In unsupervised machine learning, the processing element may be required to find its own structure in unlabeled example inputs.

### Exemplary Components, Apparatuses, and Devices

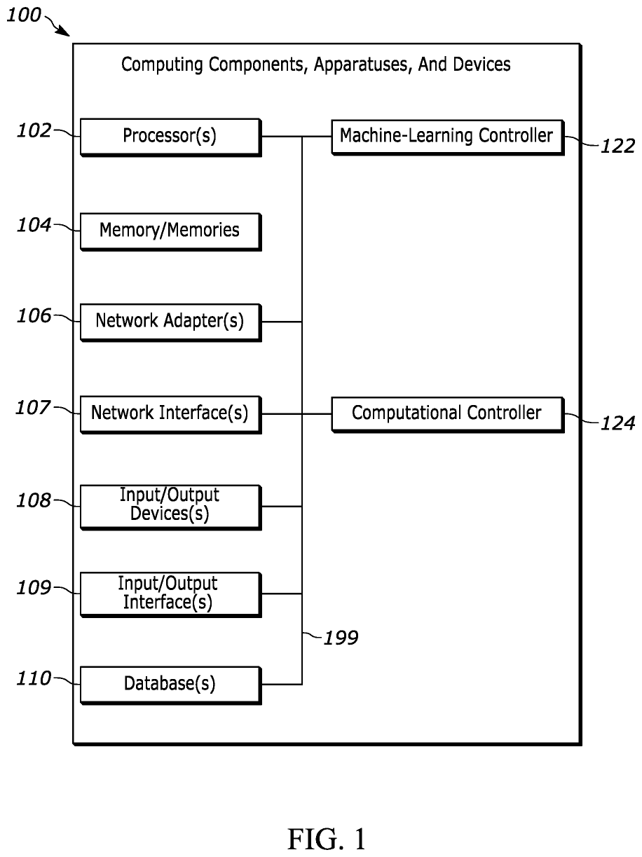

FIG. 1 depicts a block diagram of exemplary components, apparatuses, and devices 100 to predict an efficacy value of an untested pharmaceutical for treating a malady.

The exemplary components, apparatuses, and devices 100 may include one or more processors 102 (e.g., a programmable processor, a programmable controller, a GPU, a DSP, an ASIC, a PLD, an FPGA, an FPLD, etc.), one or more memories (e.g., random access memory (RAM), read only memory (ROM), cache, etc.) 104, one or more network adapters 106, one or more network interfaces 107, one or more I/O devices 108, one or more I/O interfaces 109, one or more databases 110, one or more machine-learning controllers 122, and/or one or more computational controllers 124 all of which may be interconnected via an address/data bus 199. The one or more memories 104 may store software and/or computer-executable instructions, which may be executed by the one or more processors 102.

The one or more processors 102 may be, or may include, a central processing unit (CPU), a graphical processing unit (GPU), a digital signal processor (DSP), an application specific integrated circuit (ASIC), a programmable logic device (PLD), a field-programmable gate array (FPGA), a field-programmable logic device (FPLD), etc.

The one or more memories 104 may be, or may include, any local short term memory (e.g., random access memory (RAM), read only memory (ROM), cache, etc.) and/or any long term memory (e.g., hard disk drives (HDD), solid state drives (SSD), etc.).

The one or more network adapters 106 and/or the one or more network interfaces 107 may be, or may include, a wired network adapter, connector, interface, etc. (e.g., an Ethernet network connector, an asynchronous transfer mode (ATM) network connector, a digital subscriber line (DSL) modem, a cable modem) and/or a wireless network adapter, connector, interface, etc. (e.g., a Wi-Fi connector, a Bluetooth® connector, an infrared connector, a cellular connector, etc.).

The one or more I/O devices 108 may be, or may include, any number of different types peripheral devices for either inputting data or outputting results. The peripheral devices may be any desired type of device such as a keyboard, a display (a liquid crystal display (LCD), a cathode ray tube (CRT) display, touch, etc.), a navigation device (a mouse, a trackball, a capacitive touch pad, a joystick, etc.), a speaker, a microphone, a button, a communication interface, an antenna, etc. The one or more I/O interfaces 109 may include any number of different types of input and/or output units and/or combined I/O circuits and/or components that enable the one or more processors 102 to communicate with the peripheral devices.

The one or more databases 110 may be a server or some other form of data storage device (e.g., one or more memories 104, CDs, CD-ROMs, DVDs, Blu-ray disks, etc.). In some examples, the one or more databases 110 store one or more sets of training/testing data.

The one or more machine-learning controllers 122 and/or the one or more computational controllers 124 may be, or may include, computer-readable, executable instructions that may be stored in the one or more memories 104 and/or performed by the one or more processors 102. Further, the computer-readable, executable instructions of the one or more machine-learning controllers 122, and/or the one or more computational controllers 124 may be stored on and/or performed by specifically designated hardware (e.g., micro controllers, microchips, etc.) which may have functionalities similar to the one or more memories 104 and/or the one or more processors 102.

### Exemplary Machine Learning Environments

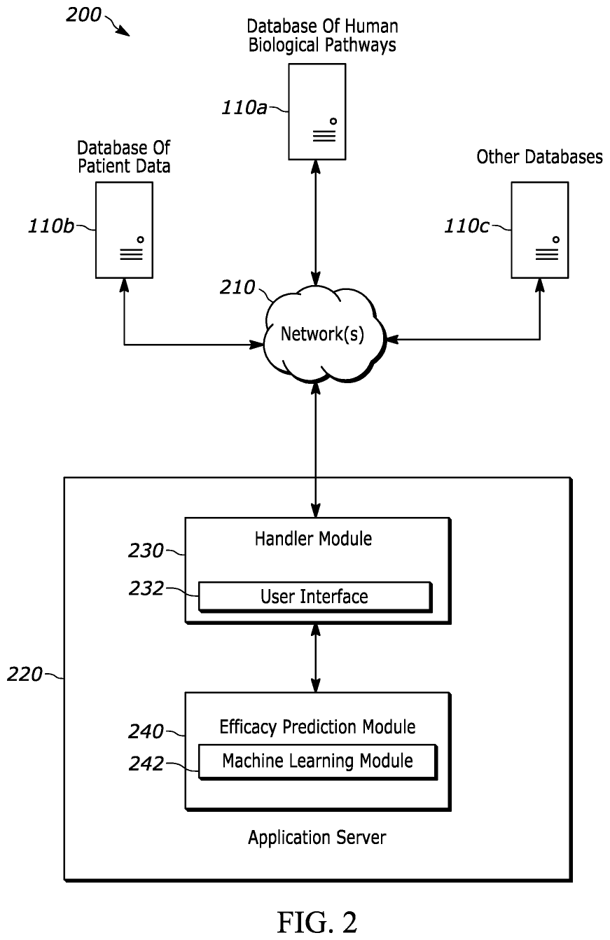

FIG. 2 depicts a diagram of an exemplary computing environment 200. The computing environment 200 may include one or more databases of patient data 110a, one or more databases of human biological pathways 110b, one or more other databases 110c, one or more networks 210, an application server 220, a handler module 230, a user interface (UI) 232, an efficacy prediction module 240, and/or a machine learning module 242.

The one or more databases of patient data 110a, the one or more databases of human biological pathways 110b, and/or the one or more other databases 110c may be, or may include, one or more databases, servers, data repositories, etc. (e.g., the one or more databases 110). The one or more networks 210 may be, or may include, the internet, a local area network (LAN), a metropolitan area network (MAN), a wide area network (WAN), a wired network, a Wi-Fi network, a cellular network, a wireless network, a private network, a virtual private network, etc.

The application server 220 may include the handler module 230, and/or the efficacy prediction module 240. The handler module 230 may include UI 232. The efficacy prediction module 240 may include a machine learning module 242. The application server 220, the handler module 230, the UI 232, the efficacy prediction module 240, and/or the machine learning module 242, may be, or may include, a portion of a memory unit (e.g., the one or more memories 104 of FIG. 1) configured to store software and/or computer-executable instructions that, when executed by a processing unit (e.g., the one or more processors 102 of FIG. 1), may cause the one or more of the aforementioned components to predict an efficacy value of an untested pharmaceutical for treating a malady.

In operation, the application server 220 may connect to the one or more databases, servers, and/or other data repositories (e.g., one or more databases of patient data 110a, the one or more databases of human biological pathways 110b, and/or the one or more other databases 110c, etc.) via one or more networks 210. In some embodiments, the connection may include a client device establishing a client-host connection to the application server 220. In these embodiments, client device may establish the client-host connection via an application run on the client device. In some embodiments, the connection may be through either a third party connection (e.g., an email server) or a direct peer-to-peer (P2P) connection/transmission.

The handler module 230 may receive one or more sets of input data over the one or more networks 210. The handler module 230 may forward the one or more sets of input data to the efficacy prediction module 240. The efficacy prediction module 240 may pass the one or more sets of input data through the machine learning module 242, which may generate one or more predicted efficacy values of an untested pharmaceutical for treating a malady. The one or more one or more predicted efficacy values of the untested pharmaceutical may be returned to the handler module 230 which may in turn present the predicted efficacy value to a user of the client device.

In some embodiments, the handler module 230 may implement an interactive UI 232 (e.g., a web-based interface, mobile application, etc.) that may be used by the user of the client device to receive one or more one or more predicted efficacy values of an untested pharmaceutical for treating a malady.

The machine learning module 242 may generate a machine learning model based upon training data. The training data may include a set of pharmaceutical-pathway weight impact scores, a set of previously human tested pharmaceutical data, a set of human biological molecule-protein pathways, and/or a set of patient data. The set of pharmaceutical-pathway weight impact scores may include correlations (e.g., sets, lists, matrices, tables, etc.) illustrating the relationship between the set of previously human tested pharmaceutical data and the set of human biological molecule-protein pathways, wherein the correlation may be a numerical value of a previously human tested pharmaceutical data and a human biological molecule-protein pathway. The set of patient data may include a set of characteristics of each patient who took one or more previously human tested pharmaceuticals in the set of previously human tested pharmaceuticals (e.g., demographics of the patient, medical history of the patient, progression of a malady before and/or after taking the one or more previously human tested pharmaceuticals, dosage and/or duration of time of taking the one or more previously human tested pharmaceuticals, reported symptoms after taking the one or more previously human tested pharmaceuticals, etc.) as well as one or more observed clinical outcomes following the administration of one or more pharmaceuticals in treating a malady. In some embodiments, the one or more observed clinical outcomes may be experiences observed in the patients who were treated with the one or more previously human tested pharmaceuticals (e.g., years of survival). In many embodiments, these clinical outcomes are the resulting outputs predicted by the generated machine learning models. From these one or more observed clinical outcomes, an efficacy value of the one or more previously human tested pharmaceuticals may be inferred, estimated, and/or determined. The efficacy of the one or more previously human tested pharmaceuticals may be the difference in observed clinical outcomes across multiple patients treated by the one or more previously human tested pharmaceuticals (e.g., a noted increase in years of survival). This estimation of a previously human tested pharmaceutical may be determined by taking two predicted clinical outcomes, each featuring the same patient characteristics, with the first predicted clinical outcome having a first patient who took the previously human tested pharmaceutical (designating with as “Yes” or “1”) and a second predicted clinical outcome having a first patient who did not take the previously human tested pharmaceutical (designating with as “No” or “0”), and subtracting the two resulting clinical outcomes to see if the value is positive thereby indicating an increase in effectiveness (e.g., the patient who took the previously human tested pharmaceutical survived longer than the patient who did not take the previously human tested pharmaceutical) or neutral or negative thereby indicating no increase in effectiveness.

The machine learning module 242 may classify the data for each previously human tested pharmaceutical into one of several subsets of training data. For example, some subsets may be derived based upon if a previously human tested pharmaceutical was effective at any degree to one or more human biological molecule-protein pathways while some other subsets may be derived based upon the degree the previously human tested pharmaceutical affects the one or more human biological molecule-protein pathways, and yet other subsets may be derived based upon the one or more observed clinical outcomes the previously human tested pharmaceuticals have in treating the malady on each patients from the set of patients, and so on. The machine learning module 242 may analyze each of the subsets to generate the machine learning model for predicting the efficacy of an untested pharmaceutical using one or more machine learning techniques. In some aspects, other types of machine learning techniques may be adapted to solve some aspects of the presently described techniques, such as gradient boosting, neural networks, deep learning, linear regression, polynomial regression, logistic regression, support vector machines, decision trees, random forests, nearest neighbors, and/or any other suitable machine learning technique.

For example, when the machine learning technique is an ensemble of decision trees (e.g., XGBoost, Random Forest, etc.), the machine learning module 242 may collect several representative samples of each of the subsets of the training data. Using each representative sample, the machine learning module 242 may generate a decision tree for predicting the clinical outcomes of the one or more previously human tested pharmaceuticals. The machine learning module 242 may aggregate and/or combine each of the decision trees (e.g., by averaging the number of one or more observed clinical outcomes at each individual tree, calculating a weighted average, taking a majority vote, etc.) to generate the machine learning model. Each decision tree may include several nodes, branches, and leaves, where each node of the decision tree represents a test on a characteristic (e.g., one or more of: (i) the one or more observed clinical outcomes of a previously human tested pharmaceutical, (ii) the demographics of the patients who took the previously human tested pharmaceutical, (iii) the dosage of and/or treatment time of the previously human tested pharmaceutical, etc.). Each branch represents the outcome of the test. Moreover, each leaf represents a different resulting clinical outcome and confidence score attached to that potential predicted clinical outcome. Each decision tree may include any number of nodes, branches, and leaves, having any suitable number and/or types of tests on characteristics and/or statistical measures.

The resulting machine learning model may be used to determine estimated efficacy values of the previously human tested pharmaceuticals by taking the difference between the predicted clinical outcome of a patient that received a previously human tested pharmaceutical and the predicted clinical outcome of another patient who did not receive the previously human tested pharmaceutical. The resulting outputs may be averaged over a large set of patients which may in turn yield an estimated efficacy value of the previously human tested pharmaceutical across a set of patients featuring certain characteristics. This particular application of machine learning models is referred to as causal inference via G-estimation.

As another example, when the machine learning technique is linear regression analysis, one or more observed clinical outcomes of each previously human tested pharmaceutical may be dependent variables and each of the characteristics may be independent variables. The machine learning module 242 may generate a machine learning model as an equation which most closely approximates the one or more observed clinical outcomes based upon the various characteristics. In this example, the regression coefficient corresponds to one or more patient characteristics that indicate whether patients in the set of patient data who received previously human tested pharmaceuticals can be used to estimate the efficacy values of the previously human tested pharmaceuticals.

In some embodiments, an ordinary least squares method may be used to minimize the difference between the value of the predicted clinical outcome value of the previously human tested pharmaceuticals and the actual clinical outcome value of the previously human tested pharmaceuticals. Additionally, the differences between the values of each predicted clinical outcome value (ŷᵢ) using the machine learning model and the one or more observed clinical outcomes (yᵢ) may be aggregated and/or combined in any suitable manner to determine a mean square error (MSE) of the regression. The MSE may be used to determine a standard error or standard deviation (σ_{ε}) in the machine learning model, which may in turn be used to create confidence intervals. For example, assuming the data is normally distributed, a confidence interval which may include about three standard deviations from the predicted clinical outcome of the previously human tested pharmaceuticals using the machine learning model (ŷᵢ−3σ_{ε}−ŷᵢ+3σ_{ε}) may correspond to 99.5 percent confidence. A confidence interval which may include about two standard deviations from the predicted clinical outcome of the previously human tested pharmaceuticals using the machine learning model (ŷᵢ−2σ_{ε}−ŷᵢ+2σ_{ε}) may correspond to 95 percent confidence. Moreover, a confidence interval which may include about 1.5 standard deviations from the predicted clinical outcome of the previously human tested pharmaceuticals using the machine learning model (ŷᵢ−1.5σ_{ε}−ŷᵢ+1.5σ_{ε}) may correspond to 90 percent confidence.

The machine learning module 242 may test the machine learning model generated. In some embodiments, the test may be conducted using the machine learning technique used to generate the model (e.g., gradient boosting, neural networks, deep learning, linear regression, polynomial regression, support vector machines, decision trees, random forests, nearest neighbors, or any other suitable machine learning technique). Further, in some embodiments, the testing data may be from the same collection of data as the training data. In these embodiments, the training data is divided into a ratio of training data and testing data (e.g., 20% training data and 80% testing data). The training data generates the machine learning model and the testing data determines the accuracy of the model. When the machine learning module 242 is correct more than a predetermined threshold amount, the machine learning model may be used for predicting the efficacy value of an untested pharmaceutical. However, if the machine learning module 242 is not correct more than the threshold amount, the machine learning module 242 may continue obtaining sets of training data and/or testing data for further training and/or testing.

Once the predicted efficacy value of an untested pharmaceutical has been made, the efficacy prediction module 240 may return the results to the handler module 230. The handler module 230 may pass the results to the user of the client device.

It should be appreciated that while specific elements, processes, devices, and/or components are described as part of the application server 220, other elements, processes, devices and/or components are contemplated.

### Exemplary Input Data

Example input data may include input data such as a set of previously human tested pharmaceutical data, untested pharmaceutical data, a set of human biological molecule-protein pathways, a set of pharmaceutical-pathway weight impact scores, a set of patient data, untreated patient data, and/or other data.

The set of pharmaceutical-pathway weight impact scores may include correlations (e.g., sets, lists, matrices, tables, etc.) illustrating the relationship between the set of previously human tested pharmaceutical data and the set of human biological molecule-protein pathways, wherein the correlation may be a numerical value of a previously human tested pharmaceutical data and a human biological molecule-protein pathway. The correlation may represent the previously human tested pharmaceutical data's impact on the particular human biological molecule-protein pathway (also herein referred to as a “weighted impact score”).

The set of patient data may include (a) a set of characteristics of each patient suffering from the malady (e.g., demographics of the patient, medical history of the patient, progression of a malady before and/or after taking the one or more previously human tested pharmaceuticals, dosage and/or duration of time of taking the one or more previously human tested pharmaceuticals, reported symptoms after taking the one or more previously human tested pharmaceuticals, etc.), (b) a set of previously human tested pharmaceutical given to each patient, and/or (c) one or more observed clinical outcomes that can be used to estimate the efficacy of the previously human tested pharmaceutical received by each patient.

The untreated patient data may include a set of characteristics (e.g., demographics, medical history, progression of a malady, etc.). The untested pharmaceutical data may include the untested pharmaceutical's weighted impact score on each of the human biological molecule-protein pathways.

Any of the foregoing example input data may be determined by the components, apparatuses, and devices 100 and/or the computing environment 200 as determined data and/or received by the components, apparatuses, and devices 100 and/or the computing environment 200 from one or more databases, servers, and/or other data repositories (e.g., one or more databases of patient data 110a, the one or more databases of human biological pathways 110b, and/or the one or more other databases 110c, etc.) over one or more networks as received data.

The components, apparatuses, and devices 100 and/or the computing environment 200 may determine any of the aforementioned data and/or any other data based upon preexisting data. For instance, the components, apparatuses, and devices 100 and/or the computing environment 200 may determine the weighted impact score between each previously human tested pharmaceutical in a set of previously human tested pharmaceuticals and each human biological molecule-protein pathway in a set of human biological molecule-protein pathways.

As an example, the components, apparatuses, and devices 100 and/or the computing environment 200 may generate a subset of human biological molecule-protein pathways based upon real world data (e.g., from clinical testing, health organizations, etc.) received from one or more databases across one or more networks indicating that a particular malady affects a human biological molecule-protein pathway in the subset and the degree by which that human biological molecule-protein pathway is affected by the malady (e.g., if it is destroyed or completely dysfunctional a value of 100% affected may be assigned and if partially functional a value of 1 to 99% may be assigned due to the degree of functionality) (“v1”). The computing system 100 and/or the computing environment 200 may determine a list of pharmaceuticals that are known to target the human biological molecule-protein pathways in the subset and the degree by which that human biological molecule-protein pathway is affected by the pharmaceutical (“v2”). For example, if a particular previously human tested pharmaceutical targets a protein within a pathway in the set of human biological molecule-protein pathways, a score of 100% may be assigned, whereas if the targeted protein is not in the pathway itself but interacts with and/or is associated with a different protein in the pathway, a score of 1 to 99% may be assigned. Similarly, if the targeted protein is neither in the pathway nor interacts with and/or is associated with a protein within the pathway, a score of 0% may be assigned. The computing system 100 and/or the computing environment 200 may process v1 and v2 to determine the correlation based upon the impact of the pharmaceutical on each human biological molecule-protein pathway in the subset (e.g., determining the average between the values, the median between the values, the root mean square between the values, etc.). Additionally or alternatively, the components, apparatuses, and devices 100 and/or the computing environment 200 may determine the correlation based solely on either v1 or v2.

The components, apparatuses, and devices 100 and/or the computing environment 200 may receive any of the aforementioned data and/or any other data from one or more databases and/or other data repositories stored across one or more networks 210.

Any of the foregoing input data may include one or more fields, labels, entries, parameters, and/or values in addition to, interchanged with, and/or instead of those listed.

### Exemplary Input Vector Using Dummy Data

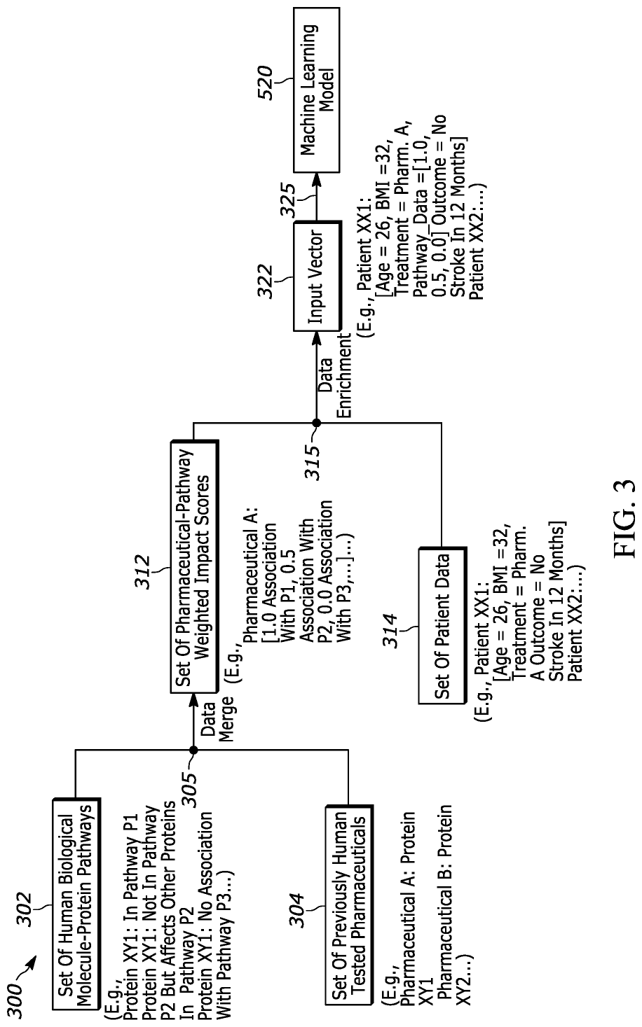

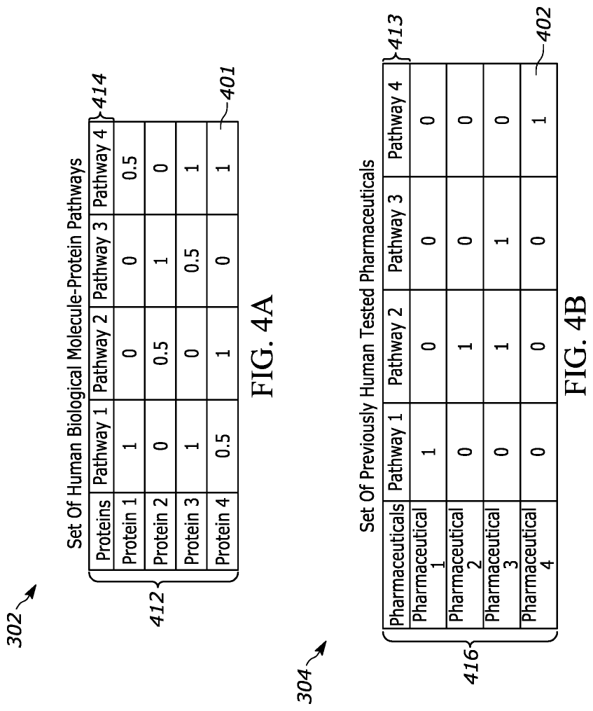

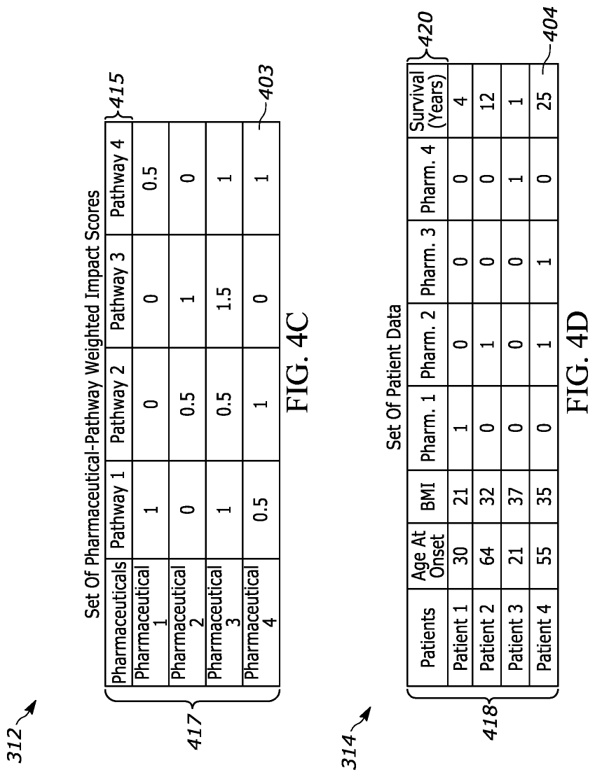

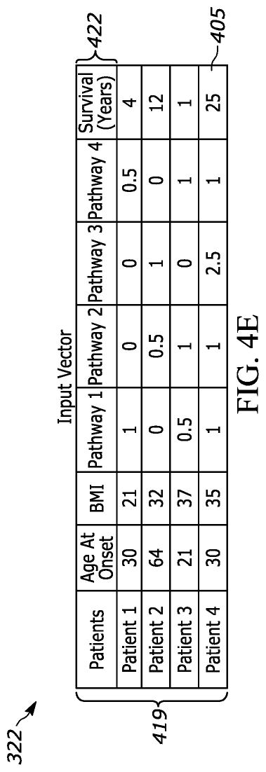

Prior to application, the untested pharmaceutical efficacy value prediction system may first generate an input vector 322 of the machine learning model 520 using the input data described above. The input vector 322 may include at least one transformed two-dimensional (2D) set (e.g., lists, tables matrices, etc.) of data (also herein referred to as “enriched data”), as illustrated in FIGS. 3-4E. In various aspects, the enriched data may comprise pre-processing, formatting, and/or normalizing data into different formats, representations, or values in order to improve the machine learning model 520, for example, by making its output more accurate and/or by reducing the data input size and/or amount needed for executing the machine learning model 520 once trained.

In some embodiments, the input vector 322 may be derived by the transformation of (i) a set of pharmaceutical-pathway weight impact scores 312 between one or more previously human tested pharmaceuticals and one or more human biological molecule-protein pathways and (ii) a set of patient data 314. The set of pharmaceutical-pathway weight impact scores 312 may be derived by the merger, normalization, formatting, or otherwise transformation of (a) a set of human biological molecule-protein pathways 302 and (b) a set of previously human tested pharmaceuticals 304.

The set of human biological molecule-protein pathways 302 may be a 2D set of data where the rows of the set of human biological molecule-protein pathways 302 indicate a set of distinct molecules and/or proteins 412 and the columns indicate a set of distinct human biological pathways 414. In some embodiments, the individual entries of the set of human biological molecule-protein pathways 302 may be a trinary entry indicating that a particular molecule and/or protein is within and/or is associated with a particular human biological pathway 401 (e.g., a value of “1” indicates that the particular molecule and/or protein is in the particular human biological pathway, a value of “0.5” indicates that the particular molecule and/or protein is not in the particular human biological pathway but is associated with a different molecule and/or protein that is in the particular human biological pathway, and a value of “0” indicates that the particular molecule and/or protein is neither in the particular human biological pathway nor is associated with a different molecule and/or protein that is in the particular human biological pathway), as illustrated in FIG. 4A. In some alternative embodiments, the individual entries of the set of human biological molecule-protein pathways 302 may be a range from 0 to 1.

The set of previously human tested pharmaceuticals 304 may be a 2D set of data where the rows of the set of previously human tested pharmaceuticals 304 indicate a set of distinct previously human tested pharmaceuticals 416 and the columns indicate a set of distinct molecules and/or proteins 413 targeted by the previously human tested pharmaceuticals. In some embodiments, the set of molecules and/or proteins 413 may be the same set as the set of molecules and/or proteins 412 in the set of human biological molecule-protein pathways 302. In some embodiments, the individual entries of the set of previously human tested pharmaceuticals 304 may be a binary entry indicating that a particular previously human tested pharmaceutical targets a particular molecule and/or protein 402 (e.g., a value of “1” indicates that the particular previously human tested pharmaceutical targets the particular molecule and/or protein and a value of “0” indicates that the particular previously human tested pharmaceutical does not target the particular molecule and/or protein), as illustrated in FIG. 4B.

The set of pharmaceutical-pathway weight impact scores 312 may be a 2D set of data where the rows of the set of pharmaceutical-pathway weight impact scores 312 indicate a set of distinct previously human tested pharmaceuticals 417 and the columns indicate a set of distinct human biological pathways 415. In some embodiments, the set of distinct previously human tested pharmaceuticals 417 may be the same as the set of distinct previously human tested pharmaceuticals 416 in the set of previously human tested pharmaceuticals 304 and/or the set of distinct human biological pathways 415 may be the same as in the set of distinct human biological pathways 414 in the set of human biological molecule-protein pathways 302. In some embodiments, the individual entries 404 of the set of pharmaceutical-pathway weight impact scores 312 may be a determined value of a particular previously human tested pharmaceutical's impact on a particular human biological pathway 403 (e.g., a weighted impact score). For example, referring to FIGS. 4A-4C, the first row of individual entries of “Pharmaceutical 1” of FIG. 4C may be determined from the data of FIGS. 4A and 4B (e.g., because Protein 1 is in Pathway 1 and is associated with a molecule and/or protein in Pathway 4, and because Pharmaceutical 1 targets Protein 1, the first individual entry of “Pharmaceutical 1” and “Pathway 1” may be “1,” the second individual entry of “Pharmaceutical 1” and “Pathway 2” may be “0,” the third individual entry of “Pharmaceutical 1” and “Pathway 3” may be “0,” and the fourth individual entry of “Pharmaceutical 1” and “Pathway 4” may be “0.5”). Similarly, the third row of individual entries of “Pharmaceutical 3” of FIG. 4C may be determined from the data of FIGS. 4A and 4B (e.g., because Pharmaceutical 3 targets both Protein 2 and Protein 3, and because Protein 2 is associated with a molecule and/or protein in Pathway 2 and is in Pathway 3 and Protein 3 is in Pathway 1 and Pathway 4 and is associated with a molecule and/or protein in Pathway 3, the first individual entry of “Pharmaceutical 3” and “Pathway 1” may be “1,” the second individual entry of “Pharmaceutical 3” and “Pathway 2” may be “0.5,” the third individual entry of “Pharmaceutical 3” and “Pathway 3” may be “1.5” derived from the summation impacts on that pathway from Protein 2 and Protein 3, and the fourth individual entry of “Pharmaceutical 1” and “Pathway 4” may be “0.5”). Put another way, the set of human biological molecule-protein pathways 302 and the set of previously human tested pharmaceuticals 304 may be merged together to generate the set of pharmaceutical-pathway impact scores 312 (305), as illustrated in FIG. 3. It should be noted that additional and/or alternative methods of determining the individual entries 404 of the set of pharmaceutical-pathway impact scores 312 may be used. For example, the maximal pathway activation of a given pharmaceutical may be selected (e.g., a value of “1” instead of “1.5” may be given for the entry of Pharmaceutical 3 and Pathway 3 since the activation value of Pharmaceutical 3 on Protein 2 would be “1” (1×1) and the activation value of Pharmaceutical 3 on Protein 2 would be “0.5” (1×0.5), and the greatest among those two activation values is 1).

In some embodiments, the data merger (305) of the set of human biological molecule-protein pathways 302 and the set of previously human tested pharmaceuticals 304 may be accomplished via a cross join query between the two sets of data.

The set of patient data 314 may be a 2D set of data where the rows of the set of patient data 314 indicate a set of distinct patients ailed by the malady 418 and the columns indicate (a) a set of distinct characteristics of the patients (e.g., demographics of the patients such as age of the onset of the malady, BMI, etc.), (b) a set of distinct previously human tested pharmaceuticals taken in treatment of the malady, and (c) a set of distinct observed clinical outcomes that can be used to estimate the efficacy of the previously human tested pharmaceutical received by the patient 420. In some embodiments, the individual entries of the set of patient data 314 may vary based upon the particular individual entry (404). For example, the individual entry of “Patient 1” and “Age at Onset” may be the derived from the real world data of the patient's age when they contracted the malady (which in this example is age 30 for Patient 1), the individual entry of “Patient 1” and “Pharm. 1” may be a binary indication that Patient 1 took Pharmaceutical 1 in treatment of the malady (which in this example is “1,” or yes), and the individual entry of “Patient 1” and “Survival (Years)” may indicate the observed clinical outcome of the patient with the treatment of Pharmaceutical 1 (which in this example is 4 years of survival), as illustrated in FIG. 4D.

In some embodiments, the set of patient data 314 may be fed into the machine learning module 242 to determine the relationship between (i) (a) the set of distinct characteristics of the patients and (b) a set of distinct previously human tested pharmaceuticals taken in treatment of the malady and (ii) (c) a set of distinct observed clinical outcomes 420 (e.g., an outcome model). In these embodiments, a machine learning model can be trained to predict observed clinical outcomes for new entries of the set of patient data 314 the machine learning model has not seen before. For example, based upon the training data and given (a) characteristics of a new patient and (b) pharmaceuticals taken by the new patient, the machine learning model can predict a clinical outcome of the new patient which may be subsequently verified (e.g., years of survival as in the illustrated example of FIG. 4D).

Additionally or alternatively, in some embodiments, an efficacy value of the set of distinct previously human tested pharmaceuticals 420 of the set of patient data 314 may be estimated using causal inference (e.g., “G-estimation”). In these embodiments, the efficacy value may be determined by taking the difference between the observed and/or predicted clinical outcomes of each of the pharmaceuticals taken by the one or more patients. For example, referring to FIG. 4D, let a Patient 5 have the following data values: their age at onset is 42, their BMI is 23, they have taken Pharmaceutical 1, and they have a Survival of 11 years. The system may assign a threshold observed clinical outcome of 10 years or greater of survival as a “1” and all other values as a “0”. Therefore, Patient 1 may have an observed clinical output of 0, Patient 2 may have an observed clinical output of 1, Patient 3 may have an observed clinical output of 0, and Patient 4 may have an observed clinical output of 1, and Patient 5 may have an observed clinical output of 1. The differences between the different observed clinical outcomes per pharmaceutical may be made to determine the efficacy value of each pharmaceutical. As an example, Patient 3 took only Pharmaceutical 4 and has an observed outcome of 0, and none of the other patients took Pharmaceutical 4; thus, Pharmaceutical 4 has an efficacy value of 0. As another example, both Patient 1 and Patient 5 took only Pharmaceutical 1, and Patient 1 has an observed outcome of 0 and Patient 5 has an observed outcome of 1; therefore, Pharmaceutical 1 has an efficacy value of 0 (e.g., one effective (or “1”) outcome minus one ineffective (or “0”) outcome implies that Pharmaceutical 1 is ineffective).

The input vector 322 may be a 2D set of data where the rows of the input vector 322 indicate a set of distinct patients ailed by the malady 419, and the columns indicate (a) a set of distinct characteristics of the patients (e.g., demographics of the patients such as age of the onset of the malady, BMI, etc.), (b) the weighted impact scores of the set of pharmaceutical-pathway weight impact scores 312, and (c) a set of distinct observed clinical outcomes that can be used to estimate the efficacy of the previously human tested pharmaceutical received by the patient 422. In some embodiments, the set of distinct patients 419 may be the same as the set of distinct patients 418 in the set of patient data 314. In some embodiments, the individual entries of (a) the set of distinct characteristics of the patients and (c) the set of distinct observed clinical outcomes may be the same as the set of distinct characteristics and/or the set of distinct observed clinical outcomes in the set of patient data 314 (405), as illustrated in FIG. 4E. Additionally or alternatively, the individual entries of (b) the weighted impact scores may be a determined value based upon the previously human tested pharmaceuticals taken by the patient and the weighted impact scores of those previously human tested pharmaceuticals across the set of human biological molecule-protein pathways 302 (405), as illustrated in FIG. 4E. For example, referring to FIGS. 4C and 4D, the first row of individual entries of “Patient 1” of FIG. 4E may be determined from the data of FIGS. 4C and 4D (e.g., because Patient 1 only took Pharmaceutical 1 and Pharmaceutical 1 has a weighted impact score of “1” for Pathway 1, “0” for Pathway 2, “0” for Pathway 3, and “0.5” for Pathway 4, the first individual entry of “Patient 1” and “Pathway 1” may be “1,” the second individual entry of “Patient 1” and “Pathway 2” may be “0,” the third individual entry of “Patient 1” and “Pathway 3” may be “0,” and the fourth individual entry of “Patient 1” and “Pathway 4” may be “0.5,”). Similarly, the fourth row of individual entries of “Patient 4” of FIG. 4E may be determined from the data of FIGS. 4C and 4D. In other words, because Patient 4 took both Pharmaceutical 2 and Pharmaceutical 3, Pharmaceutical 2 has a weighted impact score of “0” for Pathway 1, “0.5” for Pathway 2, “1” for Pathway 3, and “0” for Pathway 4, and Pharmaceutical 3 has a weighted impact score of “1” for Pathway 1, “0.5” for Pathway 2, “1.5” for Pathway 3, and “1” for Pathway 4, the first individual entry of “Patient 4” and “Pathway 1” may be “1,” the second individual entry of “Patient 1” and “Pathway 2” may be “1” (derived by the sum of both Pharmaceutical 1 and Pharmaceutical 2's impact on Pathway 2) the third individual entry of “Patient 1” and “Pharmaceutical 3” may be “2.5,” and the fourth individual entry of “Patient 1” and “Pharmaceutical 4” may be “1,”. Put another way, the set of patient data 314 may be enriched using the set of pharmaceutical-pathway weight impact scores 312 to generate enriched data the input vector 322 (315), as illustrated in FIG. 3.

Once generated, the input vector 322 may be used to train the machine learning model 520 (325) to predict the efficacy values of untested pharmaceuticals using the determined relationship between (i) (a) the set of distinct characteristics of the patients and (b) a set of distinct previously human tested pharmaceuticals taken in treatment of the malady and (ii) (c) a set of distinct observed clinical outcomes 420 of the set of patient data 314 and the determined weighted impact scores 403 of the set of pharmaceutical-pathway weighted impact scores 312 so long as the machine learning model 520 is given the weighted impact scores of the untested pharmaceutical against each of the human biological molecule-protein pathways in the set of human biological molecule-protein pathways 302. In some implementations, the input vector 322 may be further processed before used to train and/or validate the machine learning model 520. For example, dimensionality reduction may be applied to reduce the input vector into a one or more smaller subsets of vectors that represent treatments for the patients.

### Alternate Exemplary Input Vector Using Dummy Data

As another example, consider the following 2D sets of data used to generate enriched data as the input vector of the machine learning model. In this example, Example Table 1 may be a set of patient data. The rows of the Example Table 1 may represent different patients ailed by the malady, and the columns of the Example Table 1 may represent (a) different characteristics about that patient (e.g., age at onset of the malady, number of hospital admissions during that window, BMI, the values of blood laboratory tests, etc.), (b) previously human tested pharmaceuticals given to the patient, and (c) observed clinical outcome that can be used to estimate the efficacy of the previously human tested pharmaceutical received by the patient. The individual entries under each previously human tested pharmaceuticals given to the patient may contain the value 1 if the patient received the pharmaceutical, or the value 0 otherwise, as illustrated in Example Table 1 below.

Example Table 1

| | Age at | | | | | Survival |
|---|---|---|---|---|---|---|
| Patient | Onset | BMI | Pharmaceutical 1 | Pharmaceutical 2 | Pharmaceutical 3 | (Years) |
| Patient 1 | 30 | 21 | 1 | 0 | 0 | 4 |
| Patient 2 | 64 | 32 | 0 | 1 | 0 | 12 |
| Patient 3 | 21 | 37 | 1 | 1 | 0 | 1 |

Example Table 2 may be a set of pharmaceutical-pathway weight impact scores. The rows of Example Table 2 may represent previously human tested pharmaceuticals, and the columns of Example Table 2 may represent different human biological pathways from the set of human biological molecule-protein pathways. The individual entries under each human biological pathway may contain the weighted impact scores each previously human tested pharmaceutical has that particular human biological pathway (e.g., Pharmaceutical 1's weighted impact scores may be 0.2 across Pathway 1, 0.3 across Pathway 2, 0.5 across Pathway 3, and 0.6 across Pathway 4; Pharmaceutical 2's weighted impact scores may be 0.7 across Pathway 1, 0.1 across Pathway 2, 0.0 across Pathway 3, and 1.0 across Pathway 4; etc.), as illustrated in Example Table 2 below.

Example Table 2

| Pharmaceuticals | Pathway 1 | Pathway 2 | Pathway 3 | Pathway 4 |
|---|---|---|---|---|
| Pharmaceutical 1 | 0.2 | 0.3 | 0.5 | 0.6 |
| Pharmaceutical 2 | 0.7 | 0.1 | 0.0 | 1.0 |

Example Table 1 and Example Table 2 may be combined into a singular table with the columns of the previously human tested pharmaceuticals of Example Table 1 replaced with the human biological pathways, as illustrated in Example Table 3 below.

Example Table 3

| | Age at | | | | | | Survival |
|---|---|---|---|---|---|---|---|
| Patient | Onset | BMI | Pathway 1 | Pathway 2 | Pathway 3 | Pathway 4 | (Years) |
| Patient 1 | 30 | 21 | 0.2 | 0.3 | 0.5 | 0.6 | 4 |
| Patient 2 | 64 | 32 | 0.7 | 0.1 | 0.0 | 1.0 | 12 |
| Patient 3 | 21 | 37 | 0.9 | 0.4 | 0.5 | 1.6 | 1 |

In this example, because Patient 1 only took Pharmaceutical 1 (as illustrated in Example Table 1), the entries under each column of human biological pathways for Patient 1 may be the weighted impact scores of Pharmaceutical 1. Similarly, because Patient 2 only took Pharmaceutical 2 (as illustrated in Example Table 2), the entries under each column of human biological pathways for Patient 2 may be the weighted impact scores of Pharmaceutical 2.

However, because Patient 3 took both Pharmaceutical 1 and Pharmaceutical 2, the entries each column of human biological pathways for Patient 3 may be (i) the sum of each pharmaceutical's weighted impact scores (as illustrated in Example Table 3), (ii) the average of pharmaceutical's weighted impact scores, (iii) the greatest weighted impact score between the pharmaceuticals per pathway, etc.

The datasets from Example Table 1 and Example Table 2 and/or their combination in Example Table 3 may be used to train a machine learning model, which is a function F(X_1, . . . , X_p, P_1, . . . , P_n)=Y, where, given a set of characteristics of the set of patient data X_1, . . . , X_p, and the weighted impact scores of the set of pharmaceutical-pathway weight impact scores P_1, . . . , P_n, produce an estimate of what the value of the target variable Y will be for a given patient in the set of patient data receiving a previously human tested pharmaceutical represented in the set of pharmaceutical-pathway weight impact scores. The target variable can be any clinical endpoint, and/or surrogate thereof, that captures patient outcomes such as survival rate, severity of disease, and/or the result of a medical test. The machine learning model may be used on any patient the model has not applied to before.

The foregoing model may be used to: (i) predict the efficacy of a pharmaceutical that has not been observed in the training data (e.g., there were no records for patients that received this treatment), (ii) predict the effect of a combination of pharmaceuticals that have not observed in the training data (e.g., each of the treatments used to the same patient), (iii) predict the effect of a known and/or previously human tested pharmaceutical that is part of a combination of pharmaceuticals that has not observed in the training data.

### Exemplary Machine Learning Modules

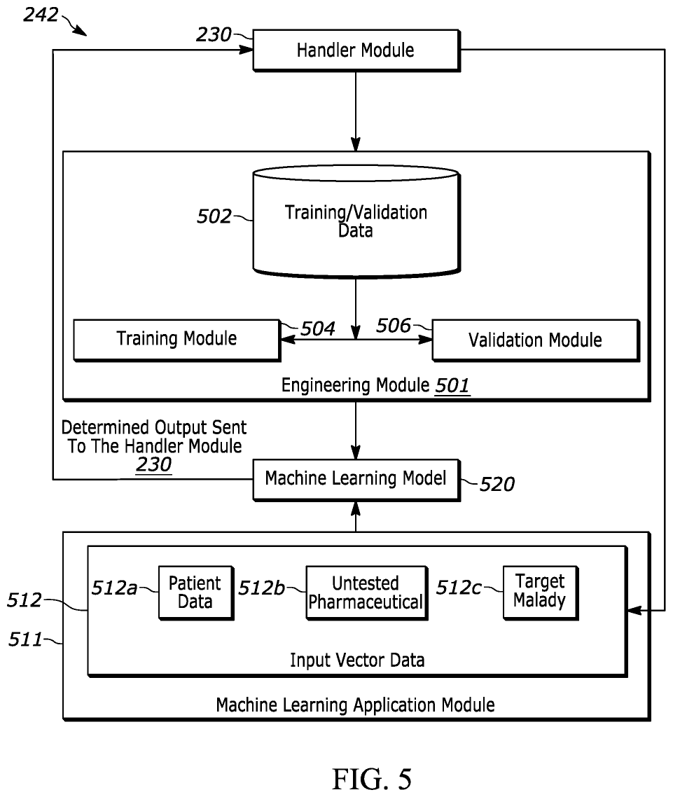

FIG. 5 depicts a diagram of exemplary machine learning modules 242. The machine learning modules 242 may include an engineering module 501, a machine learning application module 511, and a resulting machine learning model 520.

The engineering module 501 may include training and/or validation data 502, a training module 504, and/or a validation module 506. The training and/or validation data 502 may store previously human tested pharmaceuticals with one or more predicted observed clinical outcomes and/or one or more actually observed clinical outcomes which may be stored on any number or type(s) of non-transitory machine-readable storage medium or disk using any number or type(s) of data structures.

The machine learning application module 511 may deploy the trained machine learning model 520. For example, the handler module 230 may receive the exemplary input data described above and generate input vector data 512 to be input into the machine learning model 520. The input vector data 512 may include patient data 512a (e.g., the set of patient data 314 and/or a subset thereof), an untested pharmaceutical 512b, and/or one or more target maladies 512c.

The engineering module 501 and/or the machine learning application module 511, may be, or may include, a portion of a memory unit (e.g., the one or more memories 104 of FIG. 1) configured to store software and/or computer-executable instructions that, when executed by a processing unit (e.g., the one or more processors 102 of FIG. 1), may cause the one or more of the aforementioned components to generate, train, deploy, and/or validate the machine learning model 520 for predicting a clinical outcome of a previously human tested pharmaceutical and/or an untested pharmaceutical. The engineering module and/or the machine learning application module 511 may be executed for use as the machine learning module 242 of FIG. 2. There may be one or more machine learning models 520.

In operation, the handler module 230 may initially access the engineering module 501. The engineering module 501 may form input vectors from the training and/or validation data 502 which may then be passed through the training module 504 to predict clinical outcomes. Similarly, the engineering module 501 may pass previously human tested pharmaceuticals with one or more predicted observed clinical outcomes and/or one or more actually observed clinical outcomes to the validation module 506. The machine learning model 520 may be trained using supervised learning.

The validation module 506 may statistically validate the machine learning model 520, for example, by using k-fold cross-validation. In these embodiments, the training and/or validation data 502 may be randomly split into k parts, and the developing machine learning model may be trained using k−1 of the k parts of the training and/or validation data 502 which represent previously human tested pharmaceuticals with one or more predicted observed clinical outcomes and/or one or more actually observed clinical outcomes.

The developing machine learning model may be evaluated using the remaining one part of the training and/or validation data 502 which represent the previously human tested pharmaceuticals with one or more predicted observed clinical outcomes and/or one or more actually observed clinical outcomes which the machine learning model 520 has not yet been exposed to. Results of the machine learning model 520 for predicted clinical outcomes of previously human tested pharmaceuticals and/or untested pharmaceuticals are compared to the one or more predicted observed clinical outcomes and/or one or more actually observed clinical outcomes by the validation module 506 to determine the performance and/or convergence of developing machine learning model. Performance and/or convergence may be determined by, for example, identifying when a metric computed over the previously determined error rate (e.g., a mean-square metric, a rate-of-decrease metric, etc.) satisfies a criteria (e.g., a metric is less than a predetermined threshold, such as a root mean squared error).

The foregoing processes may repeat until the results of the machine learning model 520 produce a desirable error rate. The machine learning model 520 may be updated from parallel engineering modules 501. It should be appreciated that while specific elements, processes, devices, and/or components are described as part of the above exemplary machine learning modules 242, other elements, processes, devices and/or components are contemplated and/or the elements, processes, devices, and/or components may interact in different ways and/or in differing orders, etc.

### Exemplary Implementation of the Untested Pharmaceutical Efficacy Value Prediction System

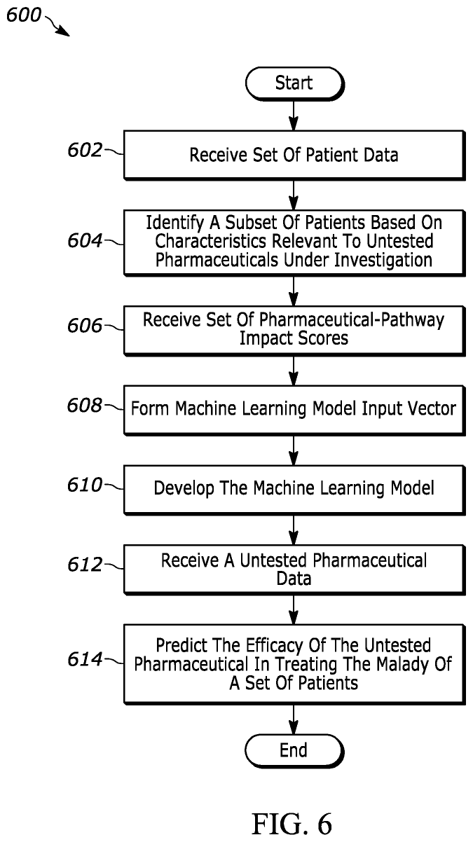

FIG. 6 depicts an exemplary computer-based method 600 for implementing predicted efficacy values of an untested pharmaceutical for treating a malady. In some aspects, the method 600 may correspond to, and/or be implemented by, the application server 220 of FIG. 2.

The processes, methods, software, and/or computer-executable instructions included within the method 600 may be, or may include, an executable program or portion of an executable program for execution by a processor such as the one or more processors 102 of FIG. 1. The program may be embodied in software or instructions stored on a non-transitory computer-readable storage medium or disk associated with the one or more processor 102. Further, although the example program is described with reference to the flowchart illustrated in FIG. 6, many other methods of implementing the application server 220 may alternatively be used. For example, the order of execution of the blocks may be changed, and/or some of the blocks described may be changed, eliminated, or combined.

Additionally, or alternatively, any or all of the blocks may be implemented by one or more hardware circuits (e.g., discrete and/or integrated analog and/or digital circuitry, an application specific integrated circuit (ASIC), a programmable logic device (PLD), a field programmable gate array (FPGA), a field programmable logic device (FPLD), a logic circuit, etc.) structured to perform the corresponding operation without executing software or firmware.

The method 600 of FIG. 6 may begin with an untested pharmaceutical efficacy value prediction system (e.g., application server 220) receiving one or more sets of patient data (e.g., the set of patient data 314 and/or a subset thereof) (block 602). The untested pharmaceutical efficacy value prediction system may then identify a subset of patients based on characteristics relevant to untested pharmaceuticals under investigation (block 604). The untested pharmaceutical efficacy value prediction system may then receive a set of pharmaceutical-pathway impact scores (block 606). The untested pharmaceutical efficacy value prediction system may then use the subset of patient data in addition to the set of pharmaceutical-pathway impact scores to form an input vector (block 608). Thus, the resulting predicted efficacy value is tailored to a specific patient ailed by the malady. The untested pharmaceutical efficacy value prediction system may use the input vector to develop a machine learning model to predict the efficacy of an untested pharmaceutical in treating a malady via a machine learning training module 500 (block 610). If the machine learning model was previously trained, the machine learning model may be retrained using the input vector. The untested pharmaceutical efficacy value prediction system may then receive untested pharmaceutical data (block 612). The untested pharmaceutical efficacy value prediction system may apply the machine learning model on the untested pharmaceutical data to predict the efficacy of the untested pharmaceutical in treating the one or more target maladies (block 614). The method 600 may exit.

### Exemplary Implementation of the Machine Learning Training Module

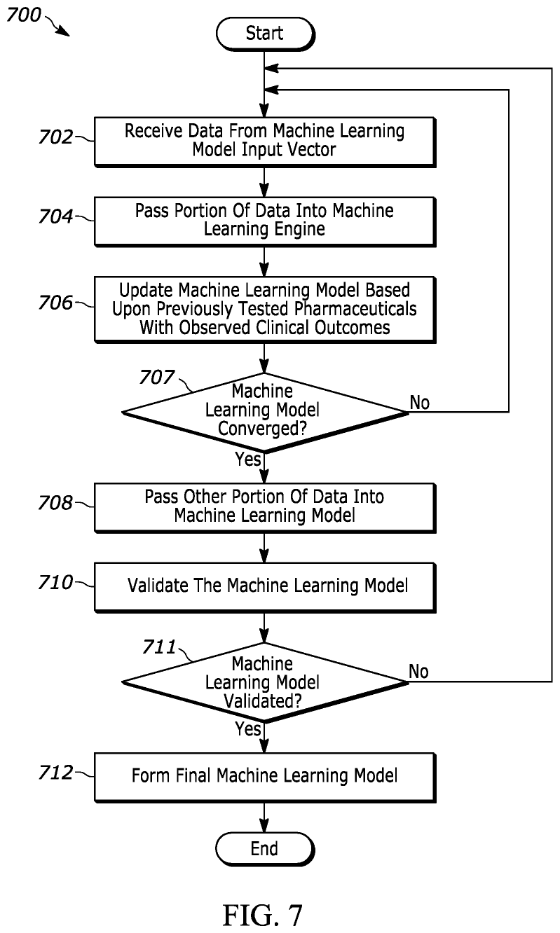

FIG. 7 depicts an exemplary computer-based method 700 for implementing the machine learning training module 700, according to some aspects. In some aspects, the method 700 may correspond to, and/or be implemented by, the training module 510, the machine learning model 520, and/or the scoring module 530 of FIG. 5.

The processes, methods, software, and/or computer-executable instructions included within the method 700 may be, or may include, an executable program or portion of an executable program for execution by a processor such as the one or more processors 102 of FIG. 1. The program may be embodied in software or instructions stored on a non-transitory computer-readable storage medium or disk associated with the one or more processor 102. Further, although the example program is described with reference to the flowchart illustrated in FIG. 7, many other methods of implementing the machine learning training module 500 may alternatively be used. For example, the order of execution of the blocks may be changed, and/or some of the blocks described may be changed, eliminated, or combined.

Additionally, or alternatively, any or all of the blocks may be implemented by one or more hardware circuits (e.g., discrete and/or integrated analog and/or digital circuitry, an application specific integrated circuit (ASIC), a programmable logic device (PLD), a field programmable gate array (FPGA), a field programmable logic device (FPLD), a logic circuit, etc.) structured to perform the corresponding operation without executing software or firmware.

The method 700 of FIG. 7 may begin when an untested pharmaceutical efficacy value prediction system receives, accesses, and/or otherwise obtains data to form an input vector (e.g., from a database storing training/testing data 512) (block 702). The method 700 may pass a portion of the data through the machine learning engine 514 (block 704). The method 700 may develop the machine learning model within the machine learning engine 514 by updating the developing machine learning model based upon comparisons between the testing module 516 and the outputs of the machine learning engine 514 (block 706).

If training of the developing machine learning model has not converged (block 707), the method 700 may return back to the start of the process to obtain more data (via block 702), and redevelop the machine learning model (via blocks 704 and 706) to continue training and developing the machine learning model. If training of the developing machine learning model has converged (block 707), the remaining portion of the data may be passed through the machine learning engine 514 (block 708). The resulting outputs of the developing machine learning model may be used by the model validation module 518 to validate the developing machine learning model (block 710).

If the model validation module 518 validates the developing machine learning model validates (block 711), the developing machine learning model may become the machine learning model 520 (block 712) that may be applied to future instances the model has not yet seen, and the method 700 may exit from the method of FIG. 7. If the model validation module 518 does not validate the developing machine learning model (block 711), the method 700 may return back to the start of the process.

### Exemplary Method

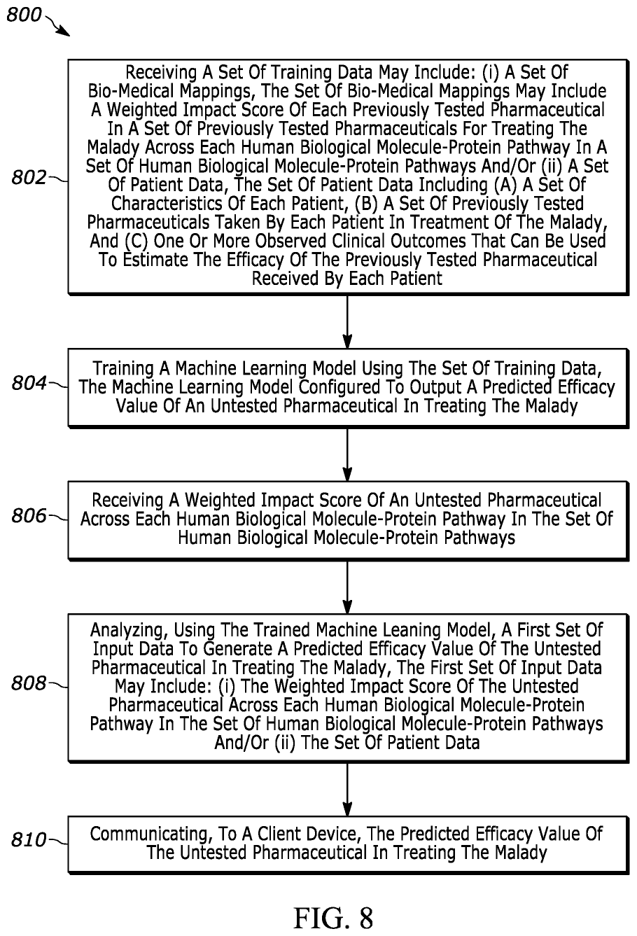

FIG. 8 depicts an exemplary computer-implemented method 800 for predicting an efficacy value of an untested pharmaceutical for treating a malady. The method 800 depicted in FIG. 8 may employ any of the techniques, methods, and systems described herein with respect to FIGS. 1-7.

The method 800 may begin at block 802 by receiving, by one or more processors, a set of training data comprising: (i) a set of pharmaceutical-pathway weight impact scores, the set of pharmaceutical-pathway weight impact scores including a weighted impact score of each previously human tested pharmaceutical in a set of previously human tested pharmaceuticals for treating the malady across each human biological molecule-protein pathway in a set of human biological molecule-protein pathways, and (ii) a set of patient data, the set of patient data including (a) a set of characteristics of each patient, (b) a set of previously human tested pharmaceuticals taken by each patient in treatment of the malady, and (c) one or more observed clinical outcomes that can be used to estimate the efficacy of the previously human tested pharmaceutical received by each patient.

The method 800 may proceed to block 804 by training, by one or more processors, a machine learning model using the set of training data, the machine learning model configured to output a predicted efficacy value of an untested pharmaceutical in treating the malady. A machine learning module (e.g., machine learning module 242) may generate a machine learning model based upon training data (e.g., the set of pharmaceutical-pathway weight impact scores and/or the set of patient data). The machine learning module may test the machine learning model generated. In some embodiments, the test may be conducted using the machine learning technique used to generate the model (e.g., gradient boosting, neural networks, deep learning, linear regression, polynomial regression, support vector machines, decision trees, random forests, nearest neighbors, or any other suitable machine learning technique). Further, in some embodiments, the testing data may be from the same collection of data as the training data. In these embodiments, the training data is divided into a ratio of training data and testing data (e.g., 20% training data and 80% testing data). The training data generates the machine learning model and the testing data determines the accuracy of the model. When the machine learning module is correct more than a predetermined threshold amount, the machine learning model may be used for predicting the efficacy of an untested pharmaceutical in treating a malady. However, if the machine learning module is not correct more than the threshold amount, the machine learning module may continue obtaining sets of training data and/or testing data for further training and/or testing.

The method 800 may proceed to block 806 by receiving, by the one or more processors, a weighted impact score of an untested pharmaceutical across each human biological molecule-protein pathway in the set of human biological molecule-protein pathways. The weighted impact score may be either received data from one or more databases across one or more networks and/or determined data by the one or more processors. The weighted impact score may be determined based upon known real world data of the untested pharmaceutical's impact on a particular human biological molecule-protein pathway and the degree by which the untested pharmaceutical impacts that human biological molecule-protein pathway. For example, if on average the untested pharmaceutical impacts a human biological molecule-protein pathway by 20% (either positively or negatively) that percentage may be treated as the weighted impact score between the untested pharmaceutical and that human biological molecule-protein pathway.

The method 800 may proceed to block 808 by analyzing, by the one or more processors using the trained machine learning model, a first set of input data to generate a predicted efficacy value of the untested pharmaceutical in treating the malady, the first set of input data comprising: (i) the weighted impact score of the untested pharmaceutical across each human biological molecule-protein pathway in the set of human biological molecule-protein pathways, and (ii) the set of patient data. The predicted efficacy value may indicate the predicted value the untested pharmaceutical will have on treating the malady based upon the trained machine learning model and the input data. The efficacy value may be used in directing research and development of pharmaceuticals and may be validated by clinical testing of the untested pharmaceutical. The validation may be fed into the machine learning model to increase its accuracy for future use.

The method 800 may proceed to block 810 by communicating, by the one or more processors to a client device, the predicted efficacy value of the untested pharmaceutical in treating the malady.

### Additional Exemplary Embodiments: Untested Pharmaceutical Efficacy Value Prediction System

In one aspect, a computer-implemented method for predicting an efficacy value of an untested pharmaceutical for treating a malady may be provided. The method may be implemented via one or more local and/or remote processors, transceivers, sensors, servers, memory units, mobile devices, wearables, smart glasses, augmented reality glasses, virtual reality headsets, and/or other electronic and/or electrical components. In one instance, the method may include: (1) receiving, by one or more processors (e.g., the one or more processors 102), a set of training data which may include: (i) a set of pharmaceutical-pathway weight impact scores, the set of pharmaceutical-pathway weight impact scores may include a weighted impact score of each previously human tested pharmaceutical in a set of previously human tested pharmaceuticals for treating the malady across each human biological molecule-protein pathway in a set of human biological molecule-protein pathways and/or (ii) a set of patient data, the set of patient data may include (a) a set of characteristics of each patient, (b) a set of previously human tested pharmaceuticals taken by each patient in treatment of the malady, and/or (c) one or more observed clinical outcomes that can be used to estimate the efficacy of the previously human tested pharmaceutical received by each patient; (2) training, by one or more processors, a machine learning model using the set of training data, the machine learning model configured to output a predicted efficacy value of an untested pharmaceutical in treating the malady; (3) receiving, by the one or more processors, a weighted impact score of an untested pharmaceutical across each human biological molecule-protein pathway in the set of human biological molecule-protein pathways; (4) analyzing, by the one or more processors using the trained machine learning model, a first set of input data to generate a predicted efficacy value of the untested pharmaceutical in treating the malady, the first set of input data may include: (i) the weighted impact score of the untested pharmaceutical across each human biological molecule-protein pathway in the set of human biological molecule-protein pathways and/or (ii) the set of patient data; and/or (5) communicating, by the one or more processors to a client device, the predicted efficacy value of the untested pharmaceutical in treating the malady. The method may include additional, less, or alternate actions, including those discussed elsewhere herein.

For instance, additionally or alternatively to the foregoing method, the predicted efficacy value of the untested pharmaceutical in treating the malady may be used (i) in a manufacturing process of the untested pharmaceutical and/or (ii) to guide a testing regimen of the untested pharmaceutical. Additionally or alternatively to the foregoing method, the method may further include altering a molecular structure and/or a proteinaceous structure of the untested pharmaceutical based upon the predicted efficacy value of the untested pharmaceutical in treating the malady. In some embodiments, this altered molecular structure and/or proteinaceous structure of the untested pharmaceutical may be used (i) in a manufacturing process of the untested pharmaceutical and/or (ii) to guide a testing regimen of the untested pharmaceutical.

Additionally or alternatively to the foregoing method, the weighted impact score may be based upon a previously human tested pharmaceutical's impact on a human biological molecule-protein pathway. In some embodiments, the method may further include determining, by the one or more processors, the weighted impact score of each previously human tested pharmaceutical in a set of previously human tested pharmaceuticals for treating the malady across each human biological molecule-protein pathway in a set of human biological molecule-protein pathways; and/or determining, by the one or more processors, the weighted impact score of the untested pharmaceutical across each human biological molecule-protein pathway in the set of human biological molecule-protein pathways.

Additionally or alternatively to the foregoing method, the method may further include determining, by the one or more processors, the set of characteristics of each patient who took the one or more previously human tested pharmaceuticals in the set of previously human tested pharmaceuticals, the set of characteristics may include one or more of: (i) demographics of the patient, (ii) medical history of the patient prior to taking the one or more previously human tested pharmaceuticals, (iii) progression of the malady after taking the one or more previously human tested pharmaceuticals, (iv) duration of time of taking the one or more previously human tested pharmaceuticals, (iv) dosage of the one or more previously human tested pharmaceuticals, and/or (v) reported symptoms after taking the one or more previously human tested pharmaceuticals. In some embodiments, the method may further include receiving, by the one or more processors, untreated patient data relating to an untreated patient currently diagnosed with the malady; identifying, by the one or more processors, a subset of patient data derived from the set of patient data based upon the demographics of patients similar to the untreated patient and/or the medical histories of patients similar to the untreated patient, the subset of patient data including (a) a set of characteristics of each patient in the subset of patient data, (b) a set of one or more previously human tested pharmaceuticals taken by each patient in the subset of patient data for treatment of the malady, and/or (c) one or more observed clinical outcomes that can be used to estimate the efficacy of the previously human tested pharmaceutical received by each patient in the subset of patient data; determining, by the one or more processors, the set of characteristics of each patient in the subset of patients who took the one or more previously human tested pharmaceuticals in the set of previously human tested pharmaceuticals, the set of characteristics of each patient in the subset of patients may include one or more of: (i) progression of the malady after taking the one or more previously human tested pharmaceuticals, (ii) duration of time of taking the one or more previously human tested pharmaceuticals, (iii) dosage of the one or more previously human tested pharmaceuticals, and/or (iv) reported symptoms after taking the one or more previously human tested pharmaceuticals; retraining, by the one or more processors, the machine learning model using: (i) the set of pharmaceutical-pathway weight impact scores, and/or (ii) the subset of patient data, the retrained machine learning model configured to output a predicted efficacy value of an untested pharmaceutical in treating the malady of the untreated patient; analyzing, by the one or more processors using the retrained machine learning model, a second set of input data to generate a predicted efficacy value of the untested pharmaceutical in treating the malady of the untreated patient, the second set of input data may include: (i) the weighted impact score of the untested pharmaceutical across each human biological molecule-protein pathway in the set of human biological molecule-protein pathways, and/or (ii) the untreated patient data; and/or communicating, by the one or more processors to a client device, the predicted efficacy value of the untested pharmaceutical in treating the malady of the untreated patient.

Additionally or alternatively to the foregoing method, analyzing the first set of input data may further include: generating, by the one or more processors, predicted efficacy values of two or more of the one or more untested pharmaceuticals in treating the malady; comparing, by the one or more processors, the efficacy values of the two or more untested pharmaceuticals; and/or communicating, by the one or more processors to a client device, the predicted efficacy value of the one or more untested pharmaceuticals that has the greatest efficacy value among the compared efficacy values.

Additionally or alternatively to the foregoing method, training the machine learning model may further include: generating, by the one or more processors, a training predicted clinical outcome of each previously human tested pharmaceuticals based upon the set of training data; comparing, by the one or more processors, the training predicted clinical outcome of each previously human tested pharmaceutical in the set of previously human tested pharmaceuticals against an actual clinical outcome of each previously human tested pharmaceutical in the set of the previously human tested pharmaceuticals; reducing, by the one or more processors, a percent rate of error of comparing the training predicted clinical outcome against actual clinical outcome by calculating one or more of: (i) an ordinary least squares of a difference between the training predicted clinical outcome of each previously human tested pharmaceuticals in the set of previously human tested pharmaceuticals and/or the actual clinical outcome of each previously human tested pharmaceuticals in the set of previously human tested pharmaceuticals, and/or (ii) an ordinary mean square of an aggregation of a resulting output between the training predicted clinical outcome of each previously human tested pharmaceuticals in the set of previously human tested pharmaceuticals and/or the actual clinical outcome of each previously human tested pharmaceuticals in the set of previously human tested pharmaceuticals; and/or generating, by the one or more processors, a confidence score based upon one or more of: (i) the training predicted clinical outcome of each previously human tested pharmaceuticals in the set of previously human tested pharmaceuticals, (ii) the actual clinical outcome of each of the previously human tested pharmaceuticals, and/or (iii) one or more standard deviations from the resulting output.

In another aspect, a computer system for predicting an efficacy value of an untested pharmaceutical for treating a malady may be provided. The computer system may be configured to include one or more local and/or remote processors, transceivers, sensors, servers, memory units, mobile devices, wearables, smart glasses, augmented reality glasses, virtual reality headsets, and/or other electronic and/or electrical components. In one instance, the computer system may include one or more processors (e.g., the one or more processors 102); and/or a non-transitory program memory (e.g., the one or more memories 104) coupled to the one or more processors and/or storing executable instructions that, when executed by the one or more processors, cause the computer system to: (1) receive a set of training data which may include: (i) a set of pharmaceutical-pathway weight impact scores, the set of pharmaceutical-pathway weight impact scores may include a weighted impact score of each previously human tested pharmaceutical in a set of previously human tested pharmaceuticals for treating the malady across each human biological molecule-protein pathway in a set of human biological molecule-protein pathways and/or (ii) a set of patient data, the set of patient data may include (a) a set of characteristics of each patient, (b) a set of previously human tested pharmaceuticals taken by each patient in treatment of the malady, and/or (c) one or more observed clinical outcomes that can be used to estimate the efficacy of the previously human tested pharmaceutical received by each patient; (2) train a machine learning model using the set of training data, the machine learning model configured to output a predicted efficacy value of an untested pharmaceutical in treating the malady; (3) receive a weighted impact score of an untested pharmaceutical across each human biological molecule-protein pathway in the set of human biological molecule-protein pathways; (4) analyze, using the trained machine learning model, a first set of input data to generate a predicted efficacy value of the untested pharmaceutical in treating the malady, the first set of input data may include: (i) the weighted impact score of the untested pharmaceutical across each human biological molecule-protein pathway in the set of human biological molecule-protein pathways and/or (ii) the set of patient data; and/or (5) communicate, to a client device, the predicted efficacy value of the untested pharmaceutical in treating the malady. The computer system may be configured to include additional, less, or alternate functionality, including that discussed elsewhere herein.

For instance, additionally or alternatively the foregoing system, the predicted efficacy value of the untested pharmaceutical in treating the malady may be used (i) in a manufacturing process of the untested pharmaceutical and/or (ii) to guide a testing regimen of the untested pharmaceutical. Additionally or alternatively to the foregoing system, the instructions may further cause the system to: alter a molecular structure and/or a proteinaceous structure of the untested pharmaceutical based upon the predicted efficacy value of the untested pharmaceutical in treating the malady. In some embodiments, this altered molecular structure and/or proteinaceous structure of the untested pharmaceutical may be used (i) in a manufacturing process of the untested pharmaceutical and/or (ii) to guide a testing regimen of the untested pharmaceutical.

Additionally or alternatively to the foregoing system, the weighted impact score may be based upon a previously human tested pharmaceutical's impact on a human biological molecule-protein pathway. In some embodiments, the instructions may further cause the system to: determine the weighted impact score of each previously human tested pharmaceutical in a set of previously human tested pharmaceuticals for treating the malady across each human biological molecule-protein pathway in a set of human biological molecule-protein pathways; and/or determine the weighted impact score of the untested pharmaceutical across each human biological molecule-protein pathway in the set of human biological molecule-protein pathways.

Additionally or alternatively to the foregoing system, the instructions may further cause the system to: determine the set of characteristics of each patient who took the one or more previously human tested pharmaceuticals in the set of previously human tested pharmaceuticals, the set of characteristics may include one or more of: (i) demographics of the patient, (ii) medical history of the patient prior to taking the one or more previously human tested pharmaceuticals, (iii) progression of the malady after taking the one or more previously human tested pharmaceuticals, (iv) duration of time of taking the one or more previously human tested pharmaceuticals, (iv) dosage of the one or more previously human tested pharmaceuticals, and/or (v) reported symptoms after taking the one or more previously human tested pharmaceuticals. In some embodiments, the instructions may further cause the system to: receive untreated patient data relating to an untreated patient currently diagnosed with the malady; identify a subset of patient data derived from the set of patient data based upon the demographics of patients similar to the untreated patient and/or the medical histories of patients similar to the untreated patient, the subset of patient data may include (a) a set of characteristics of each patient in the subset of patient data, (b) a set of one or more previously human tested pharmaceuticals taken by each patient in the subset of patient data for treatment of the malady, and/or (c) one or more observed clinical outcomes that can be used to estimate the efficacy of the previously human tested pharmaceutical received by each patient in the subset of patient data; determine the set of characteristics of each patient in the subset of patients who took the one or more previously human tested pharmaceuticals in the set of previously human tested pharmaceuticals, the set of characteristics of each patient in the subset of patients may include one or more of: (i) progression of the malady after taking the one or more previously human tested pharmaceuticals, (ii) duration of time of taking the one or more previously human tested pharmaceuticals, (iii) dosage of the one or more previously human tested pharmaceuticals, and/or (iv) reported symptoms after taking the one or more previously human tested pharmaceuticals; retrain the machine learning model using: (i) the set of pharmaceutical-pathway weight impact scores, and/or (ii) the subset of patient data, the retrained machine learning model configured to output a predicted efficacy value of an untested pharmaceutical in treating the malady of the untreated patient; analyze, using the retrained machine learning model, a second set of input data to generate a predicted efficacy value of the untested pharmaceutical in treating the malady of the untreated patient, the set of input data may include: (i) the weighted impact score of the untested pharmaceutical across each human biological molecule-protein pathway in the set of human biological molecule-protein pathways, and/or (ii) the untreated patient data; and/or communicate, to a client device, the predicted efficacy value of the untested pharmaceutical in treating the malady of the untreated patient.

Additionally or alternatively to the foregoing system, analyzing the first set of input data may further cause the system to: generate predicted efficacy values of two or more of the one or more untested pharmaceuticals in treating the malady; compare the efficacy values of the two or more untested pharmaceuticals; and/or communicate, to a client device, the predicted efficacy value of the one or more untested pharmaceuticals that has the greatest efficacy value among the compared efficacy values.

Additionally or alternatively to the foregoing system, training the machine learning model may further cause the system to: generate a training predicted clinical outcome of each previously human tested pharmaceuticals based upon the set of training data; compare the training predicted clinical outcome of each previously human tested pharmaceutical in the set of previously human tested pharmaceuticals against an actual clinical outcome of each previously human tested pharmaceutical in the set of the previously human tested pharmaceuticals; reduce a percent rate of error of comparing the training predicted clinical outcome against actual clinical outcome by calculating one or more of: (i) an ordinary least squares of a difference between the training predicted clinical outcome of each previously human tested pharmaceuticals in the set of previously human tested pharmaceuticals and/or the actual clinical outcome of each previously human tested pharmaceuticals in the set of previously human tested pharmaceuticals, and/or (ii) an ordinary mean square of an aggregation of a resulting output between the training predicted clinical outcome of each previously human tested pharmaceuticals in the set of previously human tested pharmaceuticals and/or the actual clinical outcome of each previously human tested pharmaceuticals in the set of previously human tested pharmaceuticals; and/or generate a confidence score based upon one or more of: (i) the training predicted clinical outcome of each previously human tested pharmaceuticals in the set of previously human tested pharmaceuticals, (ii) the actual clinical outcome of each of the previously human tested pharmaceuticals, and/or (iii) one or more standard deviations from the resulting output.

In another aspect, a tangible, a non-transitory computer-readable medium may store executable instructions for predicting an efficacy value of an untested pharmaceutical for treating a malady may be provided. The executable instructions, when executed, may cause one or more processors (e.g., the one or more processors 102) to: (1) receive a set of training data may include: (i) a set of pharmaceutical-pathway weight impact scores, the set of pharmaceutical-pathway weight impact scores which may include a weighted impact score of each previously human tested pharmaceutical in a set of previously human tested pharmaceuticals for treating the malady across each human biological molecule-protein pathway in a set of human biological molecule-protein pathways and/or (ii) a set of patient data, the set of patient data including (a) a set of characteristics of each patient, (b) a set of previously human tested pharmaceuticals taken by each patient in treatment of the malady, and/or (c) one or more observed clinical outcomes that can be used to estimate the efficacy of the previously human tested pharmaceutical received by each patient; (2) train a machine learning model using the set of training data, the machine learning model configured to output a predicted efficacy value of an untested pharmaceutical in treating the malady; (3) receive a weighted impact score of an untested pharmaceutical across each human biological molecule-protein pathway in the set of human biological molecule-protein pathways; (4) analyze, using the trained machine learning model, a first set of input data to generate a predicted efficacy value of the untested pharmaceutical in treating the malady, the first set of input data may include: (i) the weighted impact score of the untested pharmaceutical across each human biological molecule-protein pathway in the set of human biological molecule-protein pathways and/or (ii) the set of patient data; and/or (5) communicate, to a client device, the predicted efficacy value of the untested pharmaceutical in treating the malady. The instructions may direct additional, less, or alternate functionality, including that discussed elsewhere herein.

For instance, additionally or alternatively the foregoing executable instructions, the predicted efficacy value of the untested pharmaceutical in treating the malady may be used (i) in a manufacturing process of the untested pharmaceutical and/or (ii) to guide a testing regimen of the untested pharmaceutical. Additionally or alternatively to the foregoing executable instructions, the executable instructions may further cause the one or more processors to: alter a molecular structure and/or a proteinaceous structure of the untested pharmaceutical based upon the predicted efficacy value of the untested pharmaceutical in treating the malady. In some embodiments, this altered molecular structure and/or proteinaceous structure of the untested pharmaceutical may be used (i) in a manufacturing process of the untested pharmaceutical and/or (ii) to guide a testing regimen of the untested pharmaceutical.

Additionally or alternatively to the foregoing executable instructions, the weighted impact score may be based upon a previously human tested pharmaceutical's impact on a human biological molecule-protein pathway. In some embodiments, the executable instructions may further cause the one or more processors to: determine the weighted impact score of each previously human tested pharmaceutical in a set of previously human tested pharmaceuticals for treating the malady across each human biological molecule-protein pathway in a set of human biological molecule-protein pathways; and/or determine the weighted impact score of the untested pharmaceutical across each human biological molecule-protein pathway in the set of human biological molecule-protein pathways.

Additionally or alternatively to the foregoing executable instructions, the executable instructions may further cause the one or more processors to: determine the set of characteristics of each patient who took the one or more previously human tested pharmaceuticals in the set of previously human tested pharmaceuticals, the set of characteristics may include one or more of: (i) demographics of the patient, (ii) medical history of the patient prior to taking the one or more previously human tested pharmaceuticals, (iii) progression of the malady after taking the one or more previously human tested pharmaceuticals, (iv) duration of time of taking the one or more previously human tested pharmaceuticals, (iv) dosage of the one or more previously human tested pharmaceuticals, and/or (v) reported symptoms after taking the one or more previously human tested pharmaceuticals. In some embodiments, the executable instructions may further cause the one or more processors to: receive untreated patient data relating to an untreated patient currently diagnosed with the malady; identify a subset of patient data derived from the set of patient data based upon the demographics of patients similar to the untreated patient and/or the medical histories of patients similar to the untreated patient, the subset of patient data may include (a) a set of characteristics of each patient in the subset of patient data, (b) a set of one or more previously human tested pharmaceuticals taken by each patient in the subset of patient data for treatment of the malady, and/or (c) one or more observed clinical outcomes that can be used to estimate the efficacy of the previously human tested pharmaceutical received by each patient in the subset of patient data; determine the set of characteristics of each patient in the subset of patients who took the one or more previously human tested pharmaceuticals in the set of previously human tested pharmaceuticals, the set of characteristics of each patient in the subset of patients may include one or more of: (i) progression of the malady after taking the one or more previously human tested pharmaceuticals, (ii) duration of time of taking the one or more previously human tested pharmaceuticals, (iii) dosage of the one or more previously human tested pharmaceuticals, and/or (iv) reported symptoms after taking the one or more previously human tested pharmaceuticals; retrain the machine learning model using: (i) the set of pharmaceutical-pathway weight impact scores, and/or (ii) the subset of patient data, the retrained machine learning model configured to output a predicted efficacy value of an untested pharmaceutical in treating the malady of the untreated patient; analyze, using the retrained machine learning model, a second set of input data to generate a predicted efficacy value of the untested pharmaceutical in treating the malady of the untreated patient, the second set of input data may include: (i) the weighted impact score of the untested pharmaceutical across each human biological molecule-protein pathway in the set of human biological molecule-protein pathways, and/or (ii) the untreated patient data; and/or communicate, to a client device, the predicted efficacy value of the untested pharmaceutical in treating the malady of the untreated patient.

Additionally or alternatively to the foregoing executable instructions, analyzing the first set of input data may further cause the one or more processors to: generate predicted efficacy values of two or more of the one or more untested pharmaceuticals in treating the malady; compare the efficacy values of the two or more untested pharmaceuticals; and/or communicate, to a client device, the predicted efficacy value of the one or more untested pharmaceuticals that has the greatest efficacy value among the compared efficacy values.

Additionally or alternatively to the foregoing executable instructions, training the machine learning model may further cause the one or more processors to: generate a training predicted clinical outcome of each previously human tested pharmaceuticals based upon the set of training data; compare the training predicted clinical outcome of each previously human tested pharmaceutical in the set of previously human tested pharmaceuticals against an actual clinical outcome of each previously human tested pharmaceutical in the set of the previously human tested pharmaceuticals; reduce a percent rate of error of comparing the training predicted clinical outcome against actual clinical outcome by calculating one or more of: (i) an ordinary least squares of a difference between the training predicted clinical outcome of each previously human tested pharmaceuticals in the set of previously human tested pharmaceuticals and/or the actual clinical outcome of each previously human tested pharmaceuticals in the set of previously human tested pharmaceuticals, and/or (ii) an ordinary mean square of an aggregation of a resulting output between the training predicted clinical outcome of each previously human tested pharmaceuticals in the set of previously human tested pharmaceuticals and/or the actual clinical outcome of each previously human tested pharmaceuticals in the set of previously human tested pharmaceuticals; and/or generate a confidence score based upon one or more of: (i) the training predicted clinical outcome of each previously human tested pharmaceuticals in the set of previously human tested pharmaceuticals, (ii) the actual clinical outcome of each of the previously human tested pharmaceuticals, and/or (iii) one or more standard deviations from the resulting output.

### Exemplary Use Cases

In some embodiments, the untested pharmaceutical efficacy value prediction system may predict an efficacy value of one or more untested pharmaceuticals in treating one or maladies. The one or more untested pharmaceuticals may be known pharmaceuticals that have not been tested in treating the one or more maladies and/or newly developed pharmaceuticals. In these embodiments, the impact of the one or more untested pharmaceuticals one the human biological molecule-protein pathways must be known and/or estimated.

Additionally or alternatively, in some embodiments, the untested pharmaceutical efficacy value prediction system may predict an efficacy value of two or more pharmaceuticals in treating one or maladies wherein at least one of the two or more pharmaceuticals is an untested pharmaceutical. In these embodiments, the untested pharmaceutical efficacy value prediction system may predict the efficacy value of the two or more pharmaceuticals based upon the impact the two or more pharmaceuticals have on the various human biological molecular-protein pathways in addition to the set of patient of data and/or subset of patient data.

Additionally or alternatively, in some embodiments, the untested pharmaceutical efficacy value prediction system may determine whether a first untested pharmaceutical and/or combination of pharmaceuticals (at least one of which is an untested pharmaceutical) is more or less effective at treating a malady than a second untested pharmaceutical and/or combination of pharmaceuticals (at least one of which is an untested pharmaceutical). The untested pharmaceutical efficacy value prediction system may achieve this by predicting the efficacy values of the one or more untested pharmaceuticals and/or combinations of pharmaceuticals and comparing the resulting predicted efficacy values to determine which of the untested pharmaceuticals and/or combinations of pharmaceuticals have the greater efficacy value.

Additionally or alternatively, in some embodiments, the untested pharmaceutical efficacy value prediction system may predict an efficacy value of one or more untested pharmaceuticals in treating one or more subsets and/or individual patients ailed by one or maladies. This may be accomplished by tailoring the machine learning model of the untested pharmaceutical efficacy value prediction system to the one or more subsets and/or individual patients by generating one or more subsets of the patient data according to similar characteristics found in the target subsets and/or individuals and training the machine learning model accordingly. For example, the machine learning model may be trained using a subset of patients who are under the age of 50, and the resulting trained machine learning model may be used to predict an efficacy value of an untested pharmaceutical in subsets of patients (and/or individuals) suffering from the one or more maladies that are also under the age of 50. As another example, the machine learning model may be trained using patient data that includes information on the age of the patients, and has learned the relationship between age and the outcomes of the patient on a given pharmaceutical.

In any of the foregoing embodiments, the predicted efficacy values may be used to guide research, development, and/or manufacturing of pharmaceuticals. For example, the predicted efficacy value may be used for predicting the outcome of potential clinical trials and/or tests (any of which may still be performed to check the accuracy of the machine learning model) which may in turn be used in determining potential alterations to the chemical makeup (e.g., the molecular structure, proteinaceous structure, etc.) of untested pharmaceutical to further develop even more effective treatments for the one or more maladies.

### ADDITIONAL CONSIDERATIONS

Although the text herein sets forth a detailed description of numerous different embodiments, it should be understood that the legal scope of the invention is defined by the words of the claims set forth at the end of this patent. The detailed description is to be construed as exemplary only and does not describe every possible embodiment, as describing every possible embodiment would be impractical, if not impossible. One could implement numerous alternate embodiments, using either current technology or technology developed after the filing date of this patent, which would still fall within the scope of the claims.

Throughout this specification, plural instances may implement components, operations, or structures described as a single instance. Although individual operations of one or more methods are illustrated and described as separate operations, one or more of the individual operations may be performed concurrently, and nothing requires that the operations be performed in the order illustrated. Structures and functionality presented as separate components in example configurations may be implemented as a combined structure or component. Similarly, structures and functionality presented as a single component may be implemented as separate components. These and other variations, modifications, additions, and improvements fall within the scope of the subject matter herein.

Additionally, some embodiments are described herein as including logic or a number of routines, subroutines, applications, or instructions. These may constitute either software (code embodied on a non-transitory, tangible machine-readable medium) or hardware. In hardware, the routines, etc., are tangible units capable of performing certain operations and may be configured or arranged in a certain manner. In example embodiments, one or more computer systems (e.g., a standalone, client or server computer system) or one or more modules of a computer system (e.g., a processor or a group of processors) may be configured by software (e.g., an application or application portion) as a module that operates to perform certain operations as described herein.

In various embodiments, a module may be implemented mechanically or electronically. Accordingly, the term “module” should be understood to encompass a tangible entity, be that an entity that is physically constructed, permanently configured (e.g., hardwired), or temporarily configured (e.g., programmed) to operate in a certain manner or to perform certain operations described herein. Considering embodiments in which modules are temporarily configured (e.g., programmed), each of the modules need not be configured or instantiated at any one instance in time. For example, where the modules comprise a general-purpose processor configured using software, the general-purpose processor may be configured as respective different modules at different times. Software may accordingly configure a processor, for example, to constitute a particular module at one instance of time and to constitute a different module at a different instance of time.

Modules may provide information to, and receive information from, other modules. Accordingly, the described modules may be regarded as being communicatively coupled. Where multiple of such modules exist contemporaneously, communications may be achieved through signal transmission (e.g., over appropriate circuits and buses) that connect the modules. In embodiments in which multiple modules are configured or instantiated at different times, communications between such modules may be achieved, for example, through the storage and retrieval of information in memory structures to which the multiple modules have access. For example, one module may perform an operation and store the output of that operation in a memory device to which it is communicatively coupled. A further module may, at a later time, access the memory device to retrieve and process the stored output. Modules may also initiate communications with input or output devices, and may operate on a resource (e.g., a collection of information).

The various operations of example methods described herein may be performed, at least partially, by one or more processors that are temporarily configured (e.g., by software) or permanently configured to perform the relevant operations. Whether temporarily or permanently configured, such processors may constitute processor-implemented modules that operate to perform one or more operations or functions. The modules referred to herein may, in some example embodiments, comprise processor-implemented modules.

Similarly, the methods or routines described herein may be at least partially processor-implemented. For example, at least some of the operations of a method may be performed by one or more processors or processor-implemented modules. The performance of certain of the operations may be distributed among the one or more processors, not only residing within a single machine, but deployed across a number of machines. In some example embodiments, the processor or processors may be located in a single location (e.g., within a home environment, an office environment or as a server farm), while in other embodiments the processors may be distributed across a number of locations.

Unless specifically stated otherwise, discussions herein using words such as “receiving,” “analyzing,” “generating,” “creating,” “storing,” “deploying,” “processing,” “computing,” “calculating,” “determining,” “presenting,” “displaying,” or the like may refer to actions or processes of a machine (e.g., a computer) that manipulates or transforms data represented as physical (e.g., electronic, magnetic, or optical) quantities within one or more memories (e.g., volatile memory, non-volatile memory, or a combination thereof), registers, or other machine components that receive, store, transmit, or display information. Some embodiments may be described using the expression “coupled” and “connected” along with their derivatives. For example, some embodiments may be described using the term “coupled” to indicate that two or more elements are in direct physical or electrical contact. The term “coupled,” however, may also mean that two or more elements are not in direct contact with each other, but yet still co-operate or interact with each other.

As used herein any reference to “some embodiments” means that a particular element, feature, structure, or characteristic described in connection with the embodiment may be included in at least one embodiment. The appearances of the phrase “some embodiments” in various places in the specification are not necessarily all referring to the same embodiment. In addition, use of the “a” or “an” are employed to describe elements and components of the embodiments herein. This is done merely for convenience and to give a general sense of the description. This description, and the claims that follow, should be read to include one or at least one and the singular also includes the plural unless it is obvious that it is meant otherwise.

As used herein, the terms “comprises,” “comprising,” “includes,” “including,” “has,” “having” or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus.

The patent claims at the end of this patent application are not intended to be construed under 35 U.S.C. § 112(f) unless traditional means-plus-function language is expressly recited, such as “means for” or “step for” language being explicitly recited in the claim(s).

This detailed description is to be construed as exemplary only and does not describe every possible embodiment, as describing every possible embodiment would be impractical, if not impossible. One could implement numerous alternate embodiments, using either current technology or technology developed after the filing date of this application. Upon reading this disclosure, those of skill in the art will appreciate still additional alternative structural and functional designs for the systems and methods disclosed herein.

Thus, while particular embodiments and applications have been illustrated and described, it is to be understood that the disclosed embodiments are not limited to the precise construction and components disclosed herein. Various modifications, changes and variations, which will be apparent to those skilled in the art, may be made in the arrangement, operation and details of the method and apparatus disclosed herein without departing from the spirit and scope defined in the appended claims.

The particular features, structures, or characteristics of any specific embodiment may be combined in any suitable manner and in any suitable combination with one or more other embodiments, including the use of selected features without corresponding use of other features. In addition, many modifications may be made to adapt a particular application, situation or material to the essential scope and spirit of the present invention. It is to be understood that other variations and modifications of the embodiments of the present invention described and illustrated herein are possible in light of the teachings herein and are to be considered part of the spirit and scope of the present invention.

While the preferred embodiments of the invention have been described, it should be understood that the invention is not so limited and modifications may be made without departing from the invention. The scope of the invention is defined by the appended claims, and all devices that come within the meaning of the claims, either literally or by equivalence, are intended to be embraced therein. It is therefore intended that the foregoing detailed description be regarded as illustrative rather than limiting, and that it be understood that it is the following claims, including all equivalents, that are intended to define the spirit and scope of this invention.

What is claimed is:

1. A computer-implemented method for predicting an efficacy value of one or more untested pharmaceuticals for treating a malady, the method comprising:
receiving, by one or more processors, a set of training data comprising: (i) a set of pharmaceutical-pathway weight impact scores, the set of pharmaceutical-pathway weight impact scores including a weighted impact score of each previously human tested pharmaceutical in a set of previously human tested pharmaceuticals for treating the malady across each human biological molecule-protein pathway in a set of human biological molecule-protein pathways and (ii) a set of patient data, the set of patient data including (a) a set of characteristics of each patient, (b) a set of previously human tested pharmaceuticals taken by each patient in treatment of the malady, and (c) one or more observed clinical outcomes that can be used to estimate the efficacy of the previously human tested pharmaceutical received by each patient;
training, by one or more processors, a machine learning model using the set of training data, the machine learning model configured to output a predicted efficacy value of one or more untested pharmaceuticals in treating the malady;
receiving, by the one or more processors, a weighted impact score of one or more untested pharmaceuticals across each human biological molecule-protein pathway in the set of human biological molecule-protein pathways;
analyzing, by the one or more processors using the trained machine learning model, a first set of input data to generate a predicted efficacy value of the one or more untested pharmaceuticals in treating the malady, the first set of input data comprising: (i) the weighted impact score of the one or more untested pharmaceuticals across each human biological molecule-protein pathway in the set of human biological molecule-protein pathways and (ii) the set of patient data; and
communicating, by the one or more processors to a client device, the predicted efficacy value of the one or more untested pharmaceuticals in treating the malady.

2. The computer-implemented method of claim 1,
wherein the set of training data comprises a training set of enriched data generated from pre-processing the set of training data, and
wherein the first set of input data comprises a first set of enriched data generated from pre-processing the first set of input data.

3. The computer-implemented method of claim 2, wherein pre-processing the set of training data comprises:
determining, by the one or more processors, a first degree of association between each molecule and/or protein targeted by a previously human tested pharmaceutical and each human biological molecule-protein pathway, wherein the set of human biological molecule-protein pathways comprises the first determined degree of association;
determining, by the one or more processors, the weighted impact score of each previously human tested pharmaceutical in a set of previously human tested pharmaceuticals for treating the malady across each human biological molecule-protein pathway in a set of human biological molecule-protein pathways based upon the first determined degree of association between the molecule and/or protein targeted by the previously human tested pharmaceutical and each human biological molecule-protein pathway, wherein the set of pharmaceutical-pathway weight impact scores comprises the determined weighted impact score of each previously human tested pharmaceutical;
determining, by the one or more processors, a relationship between (i) (a) the set of characteristics of each patient and (b) the set of previously human tested pharmaceuticals taken by each patient and (ii) (c) the one or more observed clinical outcomes that can be used to estimate the efficacy of the previously human tested pharmaceutical received by each patient; and
generating, by the one or more processors, the set of training data by enriching the set of patient data with the determined weighted impact score of each previously human tested pharmaceutical and the determined relationship.

4. The computer-implemented method of claim 3, wherein pre-processing the first set of input data comprises:
determining, by the one or more processors, a second degree of association between a molecule and/or protein targeted by the untested pharmaceutical and each human biological molecule-protein pathway, wherein the set of human biological molecule-protein pathways comprises the second determined degree of association;
determining, by the one or more processors, the weighted impact score of each untested pharmaceuticals across each human biological molecule-protein pathway in the set of human biological molecule-protein pathways based upon the second determined degree of association between the molecule and/or protein targeted by the untested pharmaceutical and each human biological molecule-protein pathway, wherein the set of pharmaceutical-pathway weight impact scores comprises the determined weighted impact score of each untested pharmaceutical; and
generating, by the one or more processors, the first set of input data by enriching the set of patient data with the determined weighted impact score of each untested pharmaceutical.

5. The computer-implemented method of claim 4, wherein the predicted efficacy value of the one or more untested pharmaceuticals is generated based upon the determined relationship and the generated first set of input data.

6. The computer-implemented method of claim 1, wherein the predicted efficacy value of the one or more untested pharmaceuticals in treating the malady is used to: (i) alter a molecular structure and/or a proteinaceous structure of the one or more untested pharmaceuticals, (ii) manufacture the one or more untested pharmaceuticals, and/or (iii) guide a testing regimen of the one or more untested pharmaceuticals.

7. The computer-implemented method of claim 1, further comprising:
receiving, by the one or more processors, two or more weighted impact scores of two or more pharmaceuticals in treating the malady across each human biological molecule-protein pathway in the set of human biological molecule-protein pathways, wherein at least one of the two or more pharmaceuticals is an untested pharmaceutical;
analyzing, by the one or more processors using the trained machine learning model, a second set of input data to generate a predicted efficacy value of the two or more pharmaceuticals, the second set of input data comprising: (i) the two or more weighted impact scores of the two or more pharmaceuticals across each human biological molecule-protein pathway in the set of human biological molecule-protein pathways and (ii) the set of patient data; and
communicating, by the one or more processors to a client device, the predicted efficacy value of the two or more pharmaceuticals.

8. The computer-implemented method of claim 1, further comprising:
receiving, by the one or more processors, two or more weighted impact scores of two or more pharmaceuticals in treating the malady across each human biological molecule-protein pathway in the set of human biological molecule-protein pathways, wherein at least one of the two or more pharmaceuticals is an untested pharmaceutical;
analyzing, by the one or more processors using the trained machine learning model, a second set of input data to generate a predicted efficacy value of the two or more pharmaceuticals, the second set of input data comprising: (i) the two or more weighted impact scores of the two or more pharmaceuticals across each human biological molecule-protein pathway in the set of human biological molecule-protein pathways and (ii) the set of patient data;
comparing, by the one or more processors, the efficacy values of the two or more untested pharmaceuticals; and
communicating, by the one or more processors to a client device, the predicted efficacy value of the two or more untested pharmaceuticals that has the greatest efficacy value among the compared efficacy values.

9. The computer-implemented method of claim 1, further comprising:
determining, by the one or more processors, the set of characteristics of each patient who took the one or more previously human tested pharmaceuticals in the set of previously human tested pharmaceuticals, the set of characteristics including one or more of: (i) demographics of the patient, (ii) medical history of the patient prior to taking the one or more previously human tested pharmaceuticals, (iii) progression of the malady after taking the one or more previously human tested pharmaceuticals, (iv) duration of time of taking the one or more previously human tested pharmaceuticals, (iv) dosage of the one or more previously human tested pharmaceuticals, or (v) reported symptoms after taking the one or more previously human tested pharmaceuticals;
receiving, by the one or more processors, untreated patient data relating to an untreated patient currently diagnosed with the malady;
identifying, by the one or more processors, a subset of patient data derived from the set of patient data based upon the demographics of patients similar to the untreated patient and the medical histories of patients similar to the untreated patient, the subset of patient data including (a) a set of characteristics of each patient in the subset of patient data, (b) a set of one or more previously human tested pharmaceuticals taken by each patient in the subset of patient data for treatment of the malady, and (c) one or more observed clinical outcomes that can be used to estimate the efficacy of the previously human tested pharmaceutical received by each patient in the subset of patient data;
determining, by the one or more processors, the set of characteristics of each patient in the subset of patients who took the one or more previously human tested pharmaceuticals in the set of previously human tested pharmaceuticals, the set of characteristics of each patient in the subset of patients including one or more of: (i) progression of the malady after taking the one or more previously human tested pharmaceuticals, (ii) duration of time of taking the one or more previously human tested pharmaceuticals, (iii) dosage of the one or more previously human tested pharmaceuticals, or (iv) reported symptoms after taking the one or more previously human tested pharmaceuticals;
retraining, by the one or more processors, the machine learning model using: (i) the set of pharmaceutical-pathway weight impact scores, and (ii) the subset of patient data, the retrained machine learning model configured to output a predicted efficacy value of the one or more untested pharmaceuticals in treating the malady of the untreated patient;
analyzing, by the one or more processors using the retrained machine learning model, a second set of input data to generate a predicted efficacy value of the one or more untested pharmaceuticals in treating the malady of the untreated patient, the second set of input data comprising: (i) the weighted impact score of the one or more untested pharmaceuticals across each human biological molecule-protein pathway in the set of human biological molecule-protein pathways, and (ii) the untreated patient data; and
communicating, by the one or more processors to a client device, the predicted efficacy value of the one or more untested pharmaceuticals in treating the malady of the untreated patient.

10. The computer-implemented method of claim 1, wherein training the machine learning model comprises:
generating, by the one or more processors, a training predicted clinical outcome of each previously human tested pharmaceuticals based upon the set of training data; and
comparing, by the one or more processors, the training predicted clinical outcome of each previously human tested pharmaceutical in the set of previously human tested pharmaceuticals against an actual clinical outcome of each previously human tested pharmaceutical in the set of the previously human tested pharmaceuticals;
reducing, by the one or more processors, a percent rate of error of comparing the training predicted clinical outcome against actual clinical outcome by calculating one or more of: (i) an ordinary least squares of a difference between the training predicted clinical outcome of each previously human tested pharmaceuticals in the set of previously human tested pharmaceuticals and the actual clinical outcome of each previously human tested pharmaceuticals in the set of previously human tested pharmaceuticals, or (ii) an ordinary mean square of an aggregation of a resulting output between the training predicted clinical outcome of each previously human tested pharmaceuticals in the set of previously human tested pharmaceuticals and the actual clinical outcome of each previously human tested pharmaceuticals in the set of previously human tested pharmaceuticals; and
generating, by the one or more processors, a confidence score based upon one or more of: (i) the training predicted clinical outcome of each previously human tested pharmaceuticals in the set of previously human tested pharmaceuticals, (ii) the actual clinical outcome of each of the previously human tested pharmaceuticals, and/or (iii) one or more standard deviations from the resulting output.

11. A computer system for predicting an efficacy value of one or more untested pharmaceuticals for treating a malady, the computer system comprising:
one or more processors; and
one or more non-transitory program memories coupled to the one or more processors, the one or more memories storing executable instructions that, when executed by the one or more processors, cause the computer system to:
receive a set of training data comprising: (i) a set of pharmaceutical-pathway weight impact scores, the set of pharmaceutical-pathway weight impact scores including a weighted impact score of each previously human tested pharmaceutical in a set of previously human tested pharmaceuticals for treating the malady across each human biological molecule-protein pathway in a set of human biological molecule-protein pathways and (ii) a set of patient data, the set of patient data including (a) a set of characteristics of each patient, (b) a set of previously human tested pharmaceuticals taken by each patient in treatment of the malady, and (c) one or more observed clinical outcomes that can be used to estimate the efficacy of the previously human tested pharmaceutical received by each patient;
train a machine learning model using the set of training data, the machine learning model configured to output a predicted efficacy value of one or more untested pharmaceuticals in treating the malady;
receive a weighted impact score of one or more untested pharmaceuticals across each human biological molecule-protein pathway in the set of human biological molecule-protein pathways;
analyze, using the trained machine learning model, a first set of input data to generate a predicted efficacy value of the one or more untested pharmaceuticals in treating the malady, the first set of input data comprising: (i) the weighted impact score of the one or more untested pharmaceuticals across each human biological molecule-protein pathway in the set of human biological molecule-protein pathways and (ii) the set of patient data; and
communicate, to a client device, the predicted efficacy value of the one or more untested pharmaceuticals in treating the malady.

12. The computer system of claim 11,
wherein the set of training data comprises a training set of enriched data generated from pre-processing the set of training data, and
wherein the first set of input data comprises a first set of enriched data generated from pre-processing the first set of input data.

13. The computer system of claim 12, wherein pre-processing the set of training data via the executable instructions to, when executed by the one or more processors, causes the computer system to:
determine a first degree of association between each molecule and/or protein targeted by a previously human tested pharmaceutical and each human biological molecule-protein pathway, wherein the set of human biological molecule-protein pathways comprises the first determined degree of association;
determine the weighted impact score of each previously human tested pharmaceutical in a set of previously human tested pharmaceuticals for treating the malady across each human biological molecule-protein pathway in a set of human biological molecule-protein pathways based upon the first determined degree of association between the molecule and/or protein targeted by the previously human tested pharmaceutical and each human biological molecule-protein pathway, wherein the set of pharmaceutical-pathway weight impact scores comprises the determined weighted impact score of each previously human tested pharmaceutical;
determine a relationship between (i) (a) the set of characteristics of each patient and (b) the set of previously human tested pharmaceuticals taken by each patient and (ii) (c) the one or more observed clinical outcomes that can be used to estimate the efficacy of the previously human tested pharmaceutical received by each patient; and
generate the set of training data by enriching the set of patient data with the determined weighted impact score of each previously human tested pharmaceutical and the determined relationship.

14. The computer system of claim 13, wherein pre-processing the first set of input data via the executable instructions to, when executed by the one or more processors, causes the computer system to:
determine a second degree of association between a molecule and/or protein targeted by the untested pharmaceutical and each human biological molecule-protein pathway, wherein the set of human biological molecule-protein pathways comprises the second determined degree of association;
determine the weighted impact score of each untested pharmaceuticals across each human biological molecule-protein pathway in the set of human biological molecule-protein pathways based upon the second determined degree of association between the molecule and/or protein targeted by the untested pharmaceutical and each human biological molecule-protein pathway, wherein the set of pharmaceutical-pathway weight impact scores comprises the determined weighted impact score of each untested pharmaceutical; and
generate the first set of input data by enriching the set of patient data with the determined weighted impact score of each untested pharmaceutical.

15. The computer system of claim 14, wherein the predicted efficacy value of the one or more untested pharmaceuticals is generated based upon the determined relationship and the generated first set of input data.

16. The computer system of claim 11, wherein the predicted efficacy value of the one or more untested pharmaceuticals in treating the malady is used to: (i) alter a molecular structure and/or a proteinaceous structure of the one or more untested pharmaceuticals, (ii) manufacture the one or more untested pharmaceuticals, and/or (iii) guide a testing regimen of the one or more untested pharmaceuticals.

17. The computer system of claim 11, wherein the executable instructions, when executed by the one or more processors, further cause the computer system to:
receive two or more weighted impact scores of two or more pharmaceuticals in treating the malady across each human biological molecule-protein pathway in the set of human biological molecule-protein pathways, wherein at least one of the two or more pharmaceuticals is an untested pharmaceutical;
analyze, using the trained machine learning model, a second set of input data to generate a predicted efficacy value of the two or more pharmaceuticals, the second set of input data comprising: (i) the two or more weighted impact scores of the two or more pharmaceuticals across each human biological molecule-protein pathway in the set of human biological molecule-protein pathways and (ii) the set of patient data; and
communicate, to a client device, the predicted efficacy value of the two or more pharmaceuticals.

18. The computer system of claim 11, wherein the executable instructions, when executed by the one or more processors, further cause the computer system to:
receive two or more weighted impact scores of two or more pharmaceuticals in treating the malady across each human biological molecule-protein pathway in the set of human biological molecule-protein pathways, wherein at least one of the two or more pharmaceuticals is an untested pharmaceutical;
analyze, using the trained machine learning model, a second set of input data to generate a predicted efficacy value of the two or more pharmaceuticals, the second set of input data comprising: (i) the two or more weighted impact scores of the two or more pharmaceuticals across each human biological molecule-protein pathway in the set of human biological molecule-protein pathways and (ii) the set of patient data;
compare the efficacy values of the two or more untested pharmaceuticals; and
communicate, to a client device, the predicted efficacy value of the two or more untested pharmaceuticals that has the greatest efficacy value among the compared efficacy values.

19. The computer system of claim 11, wherein the executable instructions, when executed by the one or more processors, further cause the computer system to:
determine the set of characteristics of each patient who took the one or more previously human tested pharmaceuticals in the set of previously human tested pharmaceuticals, the set of characteristics including one or more of: (i) demographics of the patient, (ii) medical history of the patient prior to taking the one or more previously human tested pharmaceuticals, (iii) progression of the malady after taking the one or more previously human tested pharmaceuticals, (iv) duration of time of taking the one or more previously human tested pharmaceuticals, (iv) dosage of the one or more previously human tested pharmaceuticals, or (v) reported symptoms after taking the one or more previously human tested pharmaceuticals;
receive untreated patient data relating to an untreated patient currently diagnosed with the malady;
identify a subset of patient data derived from the set of patient data based upon the demographics of patients similar to the untreated patient and the medical histories of patients similar to the untreated patient, the subset of patient data including (a) a set of characteristics of each patient in the subset of patient data, (b) a set of one or more previously human tested pharmaceuticals taken by each patient in the subset of patient data for treatment of the malady, and (c) one or more observed clinical outcomes that can be used to estimate the efficacy of the previously human tested pharmaceutical received by each patient in the subset of patient data;
determine the set of characteristics of each patient in the subset of patients who took the one or more previously human tested pharmaceuticals in the set of previously human tested pharmaceuticals, the set of characteristics of each patient in the subset of patients including one or more of: (i) progression of the malady after taking the one or more previously human tested pharmaceuticals, (ii) duration of time of taking the one or more previously human tested pharmaceuticals, (iii) dosage of the one or more previously human tested pharmaceuticals, or (iv) reported symptoms after taking the one or more previously human tested pharmaceuticals;
retrain the machine learning model using: (i) the set of pharmaceutical-pathway weight impact scores, and (ii) the subset of patient data, the retrained machine learning model configured to output a predicted efficacy value of the one or more untested pharmaceuticals in treating the malady of the untreated patient;
analyze, using the retrained machine learning model, a second set of input data to generate a predicted efficacy value of the one or more untested pharmaceuticals in treating the malady of the untreated patient, the second set of input data comprising: (i) the weighted impact score of the one or more untested pharmaceuticals across each human biological molecule-protein pathway in the set of human biological molecule-protein pathways, and (ii) the untreated patient data; and
communicate, to a client device, the predicted efficacy value of the one or more untested pharmaceuticals in treating the malady of the untreated patient.

20. A tangible, non-transitory computer-readable medium storing executable instructions for predicting an efficacy value of one or more untested pharmaceuticals for treating a malady, the instructions, when executed by one or more processors of a computer system, cause the computer system to:
receive a set of training data comprising: (i) a set of pharmaceutical-pathway weight impact scores, the set of pharmaceutical-pathway weight impact scores including a weighted impact score of each previously human tested pharmaceutical in a set of previously human tested pharmaceuticals for treating the malady across each human biological molecule-protein pathway in a set of human biological molecule-protein pathways and (ii) a set of patient data, the set of patient data including (a) a set of characteristics of each patient, (b) a set of previously human tested pharmaceuticals taken by each patient in treatment of the malady, and (c) one or more observed clinical outcomes that can be used to estimate the efficacy of the previously human tested pharmaceutical received by each patient;
train a machine learning model using the set of training data, the machine learning model configured to output a predicted efficacy value of one or more untested pharmaceuticals in treating the malady;
receive a weighted impact score of one or more untested pharmaceuticals across each human biological molecule-protein pathway in the set of human biological molecule-protein pathways;
analyze, using the trained machine learning model, a first set of input data to generate a predicted efficacy value of the one or more untested pharmaceuticals in treating the malady, the first set of input data comprising: (i) the weighted impact score of the one or more untested pharmaceuticals across each human biological molecule-protein pathway in the set of human biological molecule-protein pathways and (ii) the set of patient data; and
communicate, to a client device, the predicted efficacy value of the one or more untested pharmaceuticals in treating the malady.
